(19) United States
(12) Patent Application Publication
(10) Pub. No.: US 2024/0148507 A1
(43) Pub. Date: May 9, 2024

(54) DEVICES AND METHODS FOR ADDRESSING VALVE LEAFLET PROBLEMS

(71) Applicant: EDWARDS LIFESCIENCES CORPORATION, Irvine, CA (US)

(72) Inventors: Taylor Jacob Scheinblum, Newport Beach, CA (US); Hiroshi Okabe, Tustin, CA (US); Harish Manickam Srinimukesh, Costa Mesa, CA (US); Richard D. White, Costa Mesa, CA (US); Nikolai Brent Poulsen, Tustin, CA (US)

(21) Appl. No.: 18/413,618

(22) Filed: Jan. 16, 2024

Related U.S. Application Data
(63) Continuation of application No. PCT/US2022/037172, filed on Jul. 14, 2022.
(60) Provisional application No. 63/222,948, filed on Jul. 16, 2021.

(51) Int. Cl. A61F 2/24

(52) U.S. Cl. CPC A61F 2/2463; A61F 2/2457; A61F 2210/009; A61F 2220/0008

(57) ABSTRACT

Valvular regurgitation is addressed by implanting devices at or near a native valve to treat leaflet issues. This can be done by treating the leaflets and/or by treating one or more natural chordae tendineae (chords). Treating the leaflets can include methods and devices that bring different leaflets together, and/or inhibiting or arresting a leaflet from billowing and/or flailing into the atrium. Treating the chords can include methods and devices that shorten chords, that increase tension in the chords, that attach chords to the ventricle wall or to each other, and the like. In each of the disclosed methods and devices, coaptation is increased and/or valvular regurgitation is reduced. The disclosed devices and methods can be performed on a beating heart.

### CROSS-REFERENCE TO RELATED APPLICATION(S)

This application is a continuation of Int'l Pat. App. No. PCT/US2022/037172 filed Jul. 14, 2022 and entitled “DEVICES AND METHODS FOR ADDRESSING VALVE LEAFLET PROBLEMS,” which claims the benefit of priority to U.S. Prov. App. No. 63/222,948 filed Jul. 16, 2021 and entitled “DEVICES AND METHODS FOR ADDRESSING VALVE LEAFLET PROBLEMS.” The entire contents of each of the above applications is incorporated by reference herein in its entirety for all purposes.

### BACKGROUND

Various disease processes can impair the proper functioning of one or more of the valves of the heart. Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. Degenerative diseases can also cause a malfunction in a leaflet of the valve, which can result in regurgitation.

Valvular regurgitation can occur when the leaflets of the valve do not close completely thereby allowing blood to leak back into the prior chamber when the heart contracts. Three mechanisms by which a valve can become regurgitant or incompetent include Carpentier's type I, type II and type III malfunctions. A Carpentier's type II malfunction involves prolapse of a segment of one or both leaflets above the plane of coaptation. This is often caused by the stretching or rupturing of chordae tendineae normally connected to the leaflet.

Nearly 4 million Americans are estimated to have moderate to severe mitral valve regurgitation (“MR”), with similar numbers of individuals impacted outside of the United States. MR can result in a volume overload on the left ventricle which in turn can progress to ventricular dilation, decreased ejection performance, pulmonary hypertension, symptomatic congestive heart failure, atrial fibrillation, right ventricular dysfunction, and death. Malfunctioning valves may either be repaired or replaced. Repair typically involves the preservation and correction of the patient's own valve. Replacement typically involves replacing the patient's malfunctioning valve with a biological or mechanical substitute. The mitral valve and tricuspid valve often suffer from deformation of the leaflets that prevents the valves from closing properly and allows for regurgitation or back flow of blood from the ventricle into the atrium, which results in valvular insufficiency. Deformations in the structure or shape of the mitral valve or tricuspid valve may be repairable.

Repairing an improperly functioning mitral valve or tricuspid valve, rather than replacing the valve, is preferable in many circumstances.

### SUMMARY

According to various examples of the disclosed technology, there is disclosed devices for reducing leaflet problems/issues, such as leaflet prolapse, flail, etc.

In some implementations, the techniques described herein relate to a device for treating and/or reducing leaflet issues, such as prolapse, flail, etc., the device including: a clip implant configured to be implanted on an atrial side of a leaflet (e.g., of a prolapsing leaflet, of a flailing leaflet etc.), the clip implant configured to secure a portion of the leaflet (e.g., an excess portion of a prolapsing leaflet, etc.) to reduce leaflet prolapse, flail, and/or other leaflet issues.

In some implementations, leaflet problems/issues are addressed by way of shortening elongated natural chords in the ventricle.

In some implementations, the clip implant is configured to pull together lateral portions of the leaflet. In some implementations, the clip implant is configured to secure an excess portion of a prolapsing leaflet without excising any portion of the prolapsing leaflet. In some implementations, the clip implant includes a spacing device to fill a gap between the leaflet and another leaflet (e.g., between a prolapsing leaflet and a non-prolapsing leaflet). In some implementations, the clip implant does not include a spacing device to fill a gap between the leaflet and another leaflet (e.g., between a prolapsing leaflet and a non-prolapsing leaflet).

In some implementations, the techniques described herein relate to a device for treating a leaflet (e.g., reducing leaflet prolapse and/or flail), the device including: a first magnetic implant secured to a leaflet (e.g., a prolapsing leaflet, a flailing leaflet, etc.); and a second magnetic implant secured to a ventricle, magnetic forces between the first magnetic implant and the second magnetic implant sufficient to reduce leaflet prolapse and/or flail.

In some implementations, the magnetic forces are configured to pull the leaflet (e.g., a portion of the leaflet, etc.) towards the ventricle. In some implementations, the second magnetic implant is implanted near an apex region of the heart. In some implementations, the first magnetic implant is secured to an atrial side of the leaflet. In some implementations, the first magnetic implant is secured to a ventricular side of the leaflet. In some implementations, the first magnetic implant is secured to an edge of the leaflet. In some implementations, the first magnetic implant is secured to the leaflet by piercing the tissue of the leaflet in such a way that a first portion of the first magnetic implant is on an atrial side of the leaflet and a second portion of the first magnetic implant is on a ventricular side of the leaflet. In some implementations, the second magnetic implant is clipped to tissue of the ventricle and the magnetic forces serve to align the clip so that the magnetic forces attract the leaflet (e.g., a portion of the leaflet, etc.) down towards an apex region of the heart.

In some implementations, the techniques described herein relate to a device for treating a leaflet, the device including: a first magnetic implant secured to a leaflet (e.g., to a first leaflet, to a prolapsing leaflet, to a flailing leaflet, etc.); and a second magnetic implant secured to another leaflet (e.g., a second leaflet, a non-prolapsing leaflet, a non-flailing leaflet, etc.), magnetic forces between the first magnetic implant and the second magnetic implant sufficient to reduce leaflet prolapse, flail, and/or another issue.

In some implementations, the first magnetic implant is secured to a free edge of the first leaflet (e.g., a prolapsing leaflet, to a flailing leaflet, etc.). In some implementations, the second magnetic implant is secured to a free edge of the second leaflet (e.g., a non-prolapsing leaflet, a non-flailing leaflet, etc.). In some implementations, the second magnetic implant is secured to a belly of the second leaflet. In some implementations, the first magnetic implant is secured to a belly of the first leaflet. In some implementations, the second magnetic implant is secured to a free edge of the second leaflet. In some implementations, the second magnetic implant is secured to a belly of the second leaflet. In some implementations, the first magnetic implant is secured to a middle portion of an edge of the first leaflet and the second magnetic implant is secured to a middle portion of an edge of the second leaflet.

In some implementations, the techniques described herein relate to a device for treating one or more leaflets of a native valve, such as prolapse, flail, etc., the device including: an annular body including an annular portion configured to be anchored to an atrial side of a leaflet; and a plurality of hooks extending from the annular body toward an edge of the leaflet, the plurality of hooks configured to protrude over the leaflet to reduce leaflet issues (e.g., prolapse, flail, etc.).

In some implementations, the plurality of hooks is curved downward toward a ventricle. In some implementations, the plurality of hooks extends straight from the annular body. In some implementations, the annular body does not encircle an annulus of the native valve. In some implementations, the annular body is implanted on an annulus of the native valve. In some implementations, the plurality of hooks is evenly spaced along the annular body. In some implementations, a majority of the plurality of hooks extends from a middle portion of the annular body so that the majority of the plurality of hooks are concentrated in the middle portion of the annular body.

In some implementations, the techniques described herein relate to a device for treating a leaflet of a native valve, the device including: a spacing material configured to be implanted between an first leaflet and a second leaflet of the native valve; a first paddle coupled to the spacing material, the first paddle including a first securing mechanism to secure a portion of the first leaflet to the first paddle; and a second paddle coupled to the spacing material, the second paddle including a second securing mechanism to secure a portion of the second leaflet to the second paddle, wherein each paddle is configured to extend and to retract from the spacing material to attach to an edge of a respective leaflet, each paddle having an independently adjustable length to enable each paddle to secure a leaflet to the spacing material to reduce leaflet prolapse.

In some implementations, the first securing mechanism and the second securing mechanism each include hooks. In some implementations, the first leaflet is a prolapsing leaflet. In some implementations, the first leaflet is a flailing leaflet. In some implementations, the second leaflet is a prolapsing leaflet. In some implementations, the second leaflet is a flailing leaflet. In some implementations, a length of each paddle is independently adjusted by manipulating elements at a proximal end of a delivery device. In some implementations, the first paddle is configured to secure a middle portion of the first leaflet and the second paddle is configured to secure a middle portion of the second leaflet. In some implementations, the techniques described herein relate to a device, wherein, in a deployed configuration, an edge of the first leaflet is configured to be secured by the securing mechanism of the first paddle and an edge of the second leaflet is configured to be secured by the securing mechanism of the second paddle.

In some implementations, the techniques described herein relate to a device for treating a leaflet of a native valve, the device including: an annular body to be anchored to an annulus of the native valve; a first flange extending from the annular body toward an edge of a first leaflet of the native valve to protrude over the first leaflet; and a second flange extending from the annular body toward an edge of a second leaflet of the native valve to protrude over the second leaflet. In some implementations, one or both of the first flange and the second flange are configured to limit prolapse of a prolapsing leaflet. In some implementations, one or both of the first flange and the second flange are configured to limit flail of a flailing leaflet.

In some implementations, the annular body includes a pliable material surrounding the annular body and the first flange and the second flange are configured to be deployed by respectively advancing a first wire and a second wire of a delivery device. In some implementations, the first wire of the delivery device extends from the annular body such that the first flange includes the first wire within the pliable material and the second wire of the delivery device extends from the annular body such that the second flange includes the second wire within the pliable material. In some implementations, the first flange and the second flange are configured to be deployed by inflating the annular body using a fluid, inflation of the annular body causing pliable material of the first flange and the second flange to inflate and extend away from the annular body. In some implementations, the first flange and the second flange are each configured to extend inward away from the annulus and downward toward the ventricle to limit prolapse and/or flail of the leaflet. In some implementations, a length of the first flange is independently adjustable from a length of the second flange.

In some implementations, the techniques described herein relate to a device for treating a leaflet of a native valve, the device including: a spacing material configured to be implanted between a first leaflet and a second leaflet, the spacing material configured to provide a surface for at least one leaflet (e.g., a first leaflet, a non-prolapsing leaflet, a non-flailing leaflet, etc.) to coapt with; and a plurality of clips extending from the spacing material, the plurality of clips configured to secure a free edge of the at least one leaflet such that a portion of the at least one leaflet contacts the spacing material.

In some implementations, the spacing material is configured to extend along approximately an entire length of the free edge of the at least one leaflet. In some implementations, the spacing material is configured to substantially fill a gap between the at least one leaflet and another leaflet (e.g., a second leaflet, a prolapsing leaflet, a flailing leaflet, etc.). In some implementations, the spacing material includes a cloth with a coiled shape set material within the cloth. In some implementations, the spacing material is configured to be inflated with a fluid. In some implementations, the spacing material is configured to be curved to follow a natural curvature of the at least one leaflet.

In some implementations, the techniques described herein relate to a device for treating a leaflet of a native valve, the device including: an anchor configured to anchor the device to an atrial appendage (e.g., to the left atrial appendage LAA); and a protruding flange secured to the anchor and extending away from the anchor and the atrial appendage toward a leaflet to inhibit prolapse and/or flail of the leaflet.

In some implementations, the anchor is configured to be positioned within an ostium of the atrial appendage. In some implementations, the anchor is configured to allow fluid to pass in and out of the atrial appendage. In some implementations, the anchor is configured to inhibit passage of fluid into the atrial appendage so that the anchor acts as an atrial appendage occluder. In some implementations, the protruding flange provides a downward force on the leaflet toward a ventricle. In some implementations, the protruding flange is configured to be deployed by inflating the protruding flange with a fluid such that the protruding flange extends away from the anchor. In some implementations, the protruding flange includes a shape set material that extends away from the anchor responsive to a temperature at the atrial appendage. In some implementations, the protruding flange is configured to lie along a portion of the leaflet.

In some implementations, the techniques described herein relate to a device for treating a leaflet of a native valve, the device including: an anchor configured to anchor the device to a septum wall in an atrium; and a protruding flange secured to the anchor and extending away from the anchor toward the leaflet to inhibit prolapse and/or of the leaflet.

In some implementations, the anchor is configured to be anchored in the septum wall at a location where a delivery device delivering the device passed through the septum wall. In some implementations, the protruding flange provides a downward force on the leaflet toward a ventricle. In some implementations, the protruding flange is configured to be deployed by inflating the protruding flange with a fluid such that the protruding flange extends away from the anchor. In some implementations, the protruding flange includes a shape set material that extends away from the anchor responsive to a temperature in the atrium. In some implementations, the protruding flange is configured to lie along a portion of the leaflet.

In some implementations, the techniques described herein relate to a device for treating a leaflet of a native valve, the device including: an atrial anchor configured to anchor to a wall of an atrium; a leaflet anchor configured to anchor to a leaflet; and a shaft connected to the atrial anchor and to the leaflet anchor and extending between the atrial anchor and the leaflet anchor, the shaft configured to limit prolapse and/or flail of the leaflet.

In some implementations, the shaft includes a compressive component configured to resist upward movement of the leaflet into the atrium. In some implementations, the atrial anchor is embedded in the wall of the atrium above another leaflet (e.g., a second leaflet, a non-prolapsing leaflet, a non-flailing leaflet, etc.). In some implementations, an angle of the shaft relative to the leaflet at a point where the leaflet anchor is anchored to the leaflet is approximately perpendicular when the native valve is closed. In some implementations, the shaft is configured to provide a force downward into a ventricle to limit prolapse and/or flail of the leaflet. In some implementations, the shaft includes a compressive component to provide elastic resistance to the leaflet. In some implementations, the shaft is configured to allow the leaflet to move into a ventricle while restricting movement into the atrium. In some implementations, the shaft includes a stiff rod encased in elastic material, the elastic material being coupled to the leaflet anchor or the atrial anchor such that movement into the ventricle stretches the elastic material and movement into the atrium is inhibited by the stiff rod. In some implementations, the atrial anchor includes a stent that deploys into the wall of the atrium.

In some implementations, the techniques described herein relate to a device for treating a leaflet of a native valve, the device including: a first free-edge clipping implant configured to attach to a free edge of a first leaflet (e.g., a non-prolapsing leaflet, a non-flailing leaflet, etc.); a second free-edge clipping implant configured to attach to a free edge of a second leaflet (e.g., a prolapsing leaflet, a flailing leaflet, etc.); a cinching mechanism configured to pull the first free-edge clipping implant and second free-edge clipping implant toward the cinching mechanism; and one or more sutures joining the two or more free-edge clipping implants to the cinching mechanism, wherein activation of the cinching mechanism causes the one or more sutures to shorten causing the first and second free-edge clipping implants to approach the cinching mechanism which is configured to approximate the second leaflet and the first leaflet to reduce valvular regurgitation.

In some implementations, the cinching mechanism includes a spooling component configured to lengthen and shorten the one or more sutures relative to the cinching mechanism. In some implementations, the cinching mechanism includes a locking component configured to lock the first free-edge clipping implant and the second free-edge clipping implant in place or to lock the one or more sutures in place.

In some implementations, the techniques described herein relate to a device for treating a leaflet of a native valve, the device including: a tube for drawing in a portion of a leaflet; a cauterizing element configured to excise the portion of the leaflet; and a clip configured to clip the cauterized portion of the leaflet.

In some implementations, the tube is configured to be advanced to a ventricular side of the leaflet to draw in the portion from a ventricular side of the leaflet. In some implementations, the clip is configured to be attached to a ventricular side of the leaflet.

In some implementations, the tube is configured to be advanced to an atrial side of the leaflet to draw in the portion from an atrial side of the leaflet. In some implementations, the clip is configured to be attached to an atrial side of the leaflet.

In some implementations, the techniques described herein relate to a device for treating a native valve, the device including: a twisting element configured to be introduced into a ventricle to twist a targeted natural chord that is elongated to effectively shorten the targeted natural chord, the targeted natural chord connected to a leaflet; and a chordal implant configured to couple to the twisted natural chord to maintain the twisted natural chord in the effectively shortened configuration, thereby inhibiting prolapse and/or flail of the leaflet.

In some implementations, the chordal implant includes a spring that couples to the twisted natural chord above and below a twisted portion of the twisted natural chord. In some implementations, the chordal implant includes a clip configured to couple directly to a twisted portion of the twisted natural chord to inhibit the twisted portion from untwisting. In some implementations, the chordal implant further includes a spring that couples to the twisted natural chord above and below the twisted portion of the twisted natural chord.

In some implementations, the techniques described herein relate to a device for treating a native valve, the device including: a chordal ring implant configured to encircle one or more elongated chords and one or more normal-length chords, the chordal ring implant configured to be cinched to approximate the one or more elongated chords to the one or more normal-length chords to improve coaptation.

In some implementations, the chordal ring implant includes a wire that is configured to partially encircle the one or more elongated chords and the one or more normal-length chords. In some implementations, the chordal ring implant further includes a cloth covering that covers the wire. In some implementations, the chordal ring is in a disconnected ring configuration in a delivery configuration. In some implementations, the chordal ring is in a connected ring configuration in a deployed configuration. In some implementations, the device is configured to transition from the delivery configuration to the deployed configuration by causing chordal ring implant in the disconnected ring configuration to partially encircle the one or more elongated chords and the one or more normal-length chords and joining ends of the chordal ring implant together to form the connected ring configuration.

In some implementations, the techniques described herein relate to a device treating a native valve, the device including: a chordal clip configured to secure a gathered portion of one or more elongated chords to a side of the one or more elongated chords, the chordal clip configured to pull the one or more elongated chords to a side, to gather the one or more pulled elongated chords, and to secure the one or more gathered elongated chords to effectively shorten the one or more elongated chords.

In some implementations, the chordal clip includes a clamp configured to secure the one or more elongated chords. In some implementations, the chordal clip includes a suture configured to secure the one or more elongated chords.

In some implementations, the techniques described herein relate to a device for treating a native valve, the device including: a staple implant configured to secure a gathered portion of one or more elongated chords to a ventricle wall, the staple implant including anchors on either side of the staple implant to secure the staple implant to the ventricle wall, the staple implant configured to pull one or more elongated chords to a side and to secure the pulled elongated chords to the ventricle wall to effectively shorten the one or more elongated chords.

In some implementations, the staple implant includes a suture that extends between a first anchor and a second anchor.

Each feature, concept, or step is independent, but can be combined with any other feature, concept, or step disclosed in this application.

Other features and aspects of the disclosed technology will become apparent from the following detailed description, taken in conjunction with the accompanying drawings, which illustrate, by way of example, the features in accordance with examples of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various examples, is described in detail with reference to the following figures. The drawings are provided for purposes of illustration only and merely depict examples of the disclosed technology. These drawings are provided to facilitate the reader's understanding of the disclosed technology and should not be considered limiting of the breadth, scope, or applicability thereof. For clarity and ease of illustration, these drawings are not necessarily made to scale.

FIG. 1 illustrates a human heart to illustrate anatomical features of the heart.

FIG. 2A illustrates an example of a healthy mitral valve.

FIGS. 2B, 2C, and 2D illustrate an example of a regurgitant mitral valve.

FIG. 3 illustrates the four chambers of the heart and the apex region of the heart.

FIGS. 4A and 4B illustrate an example clip implant designed to hold an excess portion of a leaflet.

FIG. 5 illustrates an example of magnetic implants configured to pull a prolapsing or billowing leaflet towards the left ventricle.

FIGS. 6A, 6B, 6C, 6D, 6E, and 6F illustrate example magnetic implants configured to be clipped or secured to both leaflets to improve coaptation.

FIGS. 7A, 7B, and 7C illustrate an annular implant with a body and hooks extending from the body.

FIGS. 8A, 8B, 8C, and 8D illustrate implantation of an example leaflet clipping implant that includes a spacing material and paddles with lengths that are independently adjustable.

FIGS. 9A and 9B illustrate an example flanged annular implant with a body and flanges extending from the body.

FIGS. 10A and 10B illustrate a gap-filling implant configured to secure to an edge of a non-prolapsing leaflet and to provide spacing material for coaptation with the prolapsing leaflet.

FIG. 11 illustrates an LAA implant configured to be anchored in a left atrial appendage (LAA) and to protrude over the anterior leaflet to inhibit or prevent the anterior leaflet from prolapsing.

FIG. 12 illustrates a septal implant configured to be anchored in the septum between the left atrium and the right atrium and to protrude over the posterior leaflet to inhibit or prevent the posterior leaflet from prolapsing.

FIG. 13 illustrates an atrial compression implant configured to be anchored in the wall of the left atrium and anchored on or secured to a prolapsing leaflet to inhibit or prevent prolapsing of the leaflet.

FIGS. 14A, 14B, 14C, and 14D illustrate a cinching leaflet implant configured to attach to the two leaflets and to pull the leaflets toward one another.

FIGS. 15A, 15B, 15C, and 15D illustrate an example method for clipping a leaflet, which can help to reduce or prevent leaflet prolapse.

FIGS. 16A, 16B, and 16C illustrate an example device and method for reducing chord length by spooling elongated chords around a chordal or spooling implant.

FIGS. 17A and 17B illustrate a chordal ring implant configured to bundle elongated chords with normal chords.

FIGS. 18A and 18B illustrate a chordal clip configured to cinch elongated chords from the side.

FIGS. 19A and 19B illustrate a staple implant configured to gather excess portions of elongated chords and to secure to the ventricle wall to effectively shorten the elongated chords.

The figures are not intended to be exhaustive or to limit the disclosed implementations to the precise form disclosed. The disclosed technology can be practiced with modification and alteration, and the disclosed technology is limited only by the claims and the equivalents thereof.

### DETAILED DESCRIPTION OF THE EMBODIMENTS

The headings provided herein, if any, are for convenience only and do not necessarily affect the scope or meaning of the claimed embodiments.

### Overview

Studies suggest that Carpentier type II malfunction (e.g., leaflet prolapse), often referred to as “Degenerative,” “Primary” or “Organic” MR, accounts for a significant amount of MR. Surgical resectional valve repair techniques may involve cutting out (resecting) a section of the prolapsed leaflet tissue, stitching the remaining tissue together and implanting an annuloplasty ring around the annulus.

Artificial chordae tendineae (“cords”) made of expanded polytetrafluoroethylene (“ePTFE”) suture, or another suitable material, may be placed in the leaflet and secured to the heart in the left ventricle, normally to the papillary muscle.

Dr. Alfieri has demonstrated the benefit of securing the midpoint of both leaflets together creating a double orifice valve in patients with MR known as an “Edge-to-Edge” repair or an Alfieri procedure. In addition to or instead of creating the edge-to-edge relationship, to promote a larger surface of coaptation between the anterior and posterior leaflets, and thereby to promote proper valve function and limit or prevent undesirable regurgitation, sutures extending from the leaflets can be secured together to pull or to otherwise move the posterior annulus towards the anterior leaflet and/or the anterior annulus towards to posterior leaflet. This reduces the distance between the anterior annulus and the posterior annulus (or the septal-lateral distance) (e.g., by about 10%-30%). Approximating the anterior annulus and the posterior annulus in this manner can decrease the valve orifice, and thereby decrease, limit, or otherwise prevent undesirable regurgitation.

Degenerative mitral valve repair procedures can include techniques such as resectional repair, chordal implantation, and edge-to-edge repairs. Disclosed herein are various methods and devices to address leaflet problems/issues, including prolapse and/or billowing leaflets with prolapse, which may at least be partially caused by elongated chords and/or mismatched leaflets. The disclosed methods and devices can be generally classified as approaches that affect the leaflet and approaches that affect the chords. However, it is to be understood that one or more of the disclosed methods may be combined. For example, one or more approaches that affect the leaflets can be combined with one or more approaches that affect the chords. As another example, approaches that affect the leaflets can be combined and/or approaches that affect the chords can be combined. While many of the examples discussed herein describe treating prolapse, the concepts, systems, devices, implants, techniques, methods, etc. herein can be used to treat native valves and leaflets for other issues/problems beyond prolapse, such as flail and other issues. Further, the methods, techniques, treatments, etc. herein can be performed on a living animal (e.g., human, other mammal, etc.) or on a non-living simulation, such as on a cadaver, cadaver heart, simulator (e.g., with the body parts, tissue, etc. being simulated), anthropomorphic phantom, etc.

Some devices that can be used to treat a valve in a beating heart and may be used with the concepts herein are described in International Patent Application No. PCT/US2012/043761, published as WO 2013/003228 A1, and referred to herein as “the '761 PCT Application,” the entire disclosure of which is incorporated herein by reference. Various methods for repairing tissue that can be used with the concepts herein are described in the '761 PCT Application and/or in International Patent Application No. PCT/US2016/055170, published as WO 2017/059426 A1, and referred to herein as “the '170 PCT Application,” the entire disclosure of each of which is incorporated herein by reference. The method(s) in these incorporated references as applied to the concepts herein can be performed on a living animal or on a simulation, such as on a cadaver, cadaver heart, simulator (e.g., with the body parts, heart, tissue, etc. being simulated), etc. mutatis mutandis.

The disclosed methods include inserting a delivery device into a body and extending a distal end of the delivery device to a proximal side of the tissue. Advancement of the delivery device may be performed in conjunction with sonography or direct visualization (e.g., direct transblood visualization), and/or any other suitable remote visualization technique. Furthermore, one or more steps of the disclosed methods may also be performed in conjunction with any suitable remote visualization technique. With respect to the disclosed methods, one or more parts of a procedure may be monitored in conjunction with transesophageal (TEE) guidance or intracardiac echocardiography (ICE) guidance. For example, this may facilitate and direct the movement and proper positioning of the delivery device for contacting the appropriate target cardiac region and/or target cardiac tissue (e.g., a valve leaflet, a valve annulus, or any other suitable cardiac tissue). Typical procedures for use of echo guidance are set forth in Suematsu, Y., J. Thorac. Cardiovasc. Surg. 2005; 130:1348-56 (“Suematsu”), the entire disclosure of which is incorporated herein by reference.

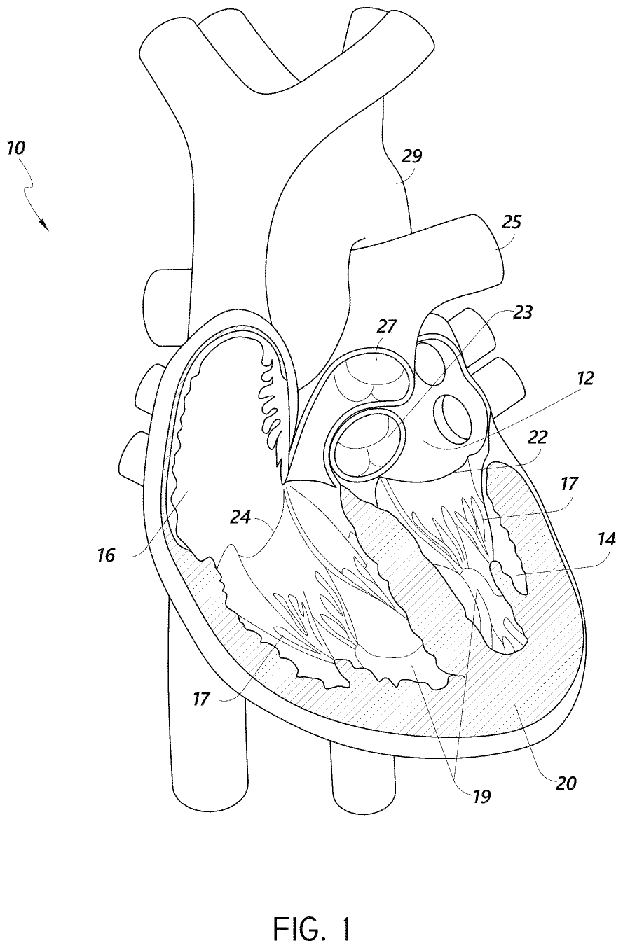

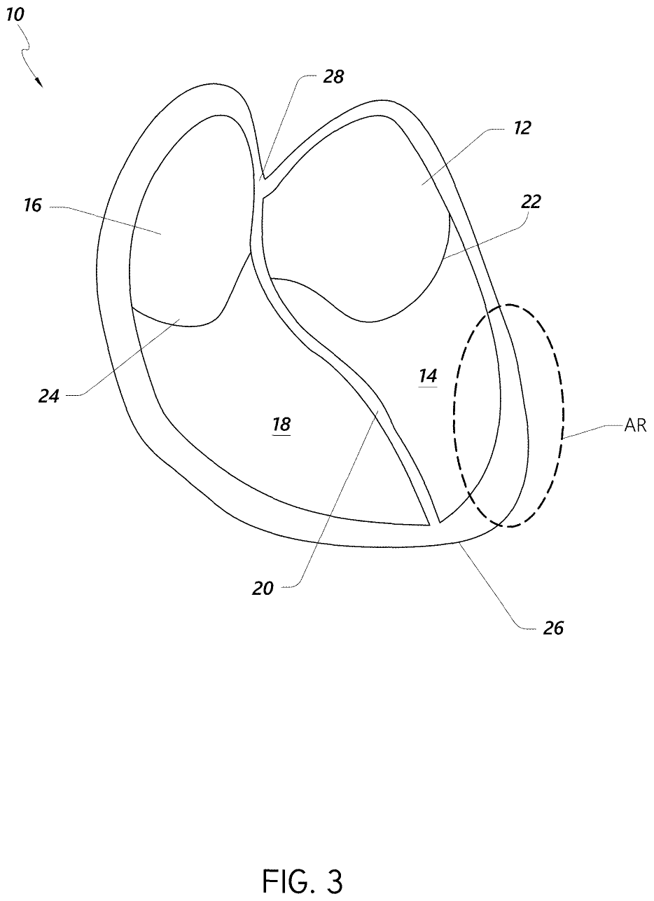

As illustrated in FIG. 1, the human heart 10 has four chambers, which include two upper chambers denoted as atria 12, 16 and two lower chambers denoted as ventricles 14, 18. A septum 20 (see, e.g., FIG. 3) divides the heart 10 and separates the left atrium 12 and left ventricle 14 from the right atrium 16 and right ventricle 18. The heart further contains four valves 22, 23, 24, and 27. The valves function to maintain the pressure and unidirectional flow of blood through the body and to prevent blood from leaking back into a chamber from which it has been pumped.

Two valves separate the atria 12, 16 from the ventricles 14, 18, denoted as atrioventricular valves. The mitral valve 22, also known as the left atrioventricular valve, controls the passage of oxygenated blood from the left atrium 12 to the left ventricle 14. A second valve, the aortic valve 23, separates the left ventricle 14 from the aortic artery (aorta) 29, which delivers oxygenated blood via the circulation to the entire body. The aortic valve 23 and mitral valve 22 are part of the “left” heart, which controls the flow of oxygen-rich blood from the lungs to the body. The right atrioventricular valve, the tricuspid valve 24, controls passage of deoxygenated blood into the right ventricle 18. A fourth valve, the pulmonary valve 27, separates the right ventricle 18 from the pulmonary artery 25. The right ventricle 18 pumps deoxygenated blood through the pulmonary artery 25 to the lungs wherein the blood is oxygenated and then delivered to the left atrium 12 via the pulmonary vein. Accordingly, the tricuspid valve 24 and pulmonic valve 27 are part of the right heart, which controls the flow of oxygen-depleted blood from the body to the lungs.

Both the left and right ventricles 14, 18 constitute pumping chambers. The aortic valve 23 and pulmonic valve 27 lie between a pumping chamber (ventricle) and a major artery and control the flow of blood out of the ventricles and into the circulation. The aortic valve 23 and pulmonic valve 27 have three cusps, or leaflets, that open and close and thereby function to prevent blood from leaking back into the ventricles after being ejected into the lungs or aorta 29 for circulation.

Both the left and right atria 12, 16 are receiving chambers. The mitral valve 22 and tricuspid valve 24, therefore, lie between a receiving chamber (atrium) and a ventricle to control the flow of blood from the atria to the ventricles and to prevent blood from leaking back into the atrium during ejection from the ventricle. Both the mitral valve 22 and tricuspid valve 24 include two or more cusps, or leaflets (not shown in FIG. 1), that are encircled by a variably dense fibrous ring of tissues known as the annulus (not shown in FIG. 1). The valves are anchored to the walls of the ventricles by chordae tendineae (chordae) 17. The chordae tendineae 17 are cord-like tendons that connect the papillary muscles 19 to the leaflets (not shown in FIG. 1) of the mitral valve 22 and tricuspid valve 24 of the heart 10. The papillary muscles 19 are located at the base of the chordae tendineae 17 and are within the walls of the ventricles. The papillary muscles 19 do not open or close the valves of the heart, which close passively in response to pressure gradients; rather, the papillary muscles 19 brace the valves against the high pressure needed to circulate the blood throughout the body. Together, the papillary muscles 19 and the chordae tendineae 17 are known as the sub-valvular apparatus. The function of the sub-valvular apparatus is to keep the valves from prolapsing into the atria when they close.

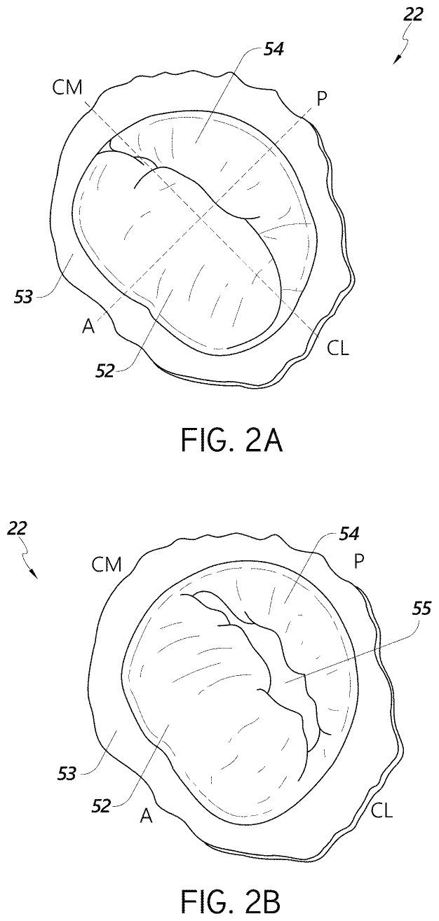

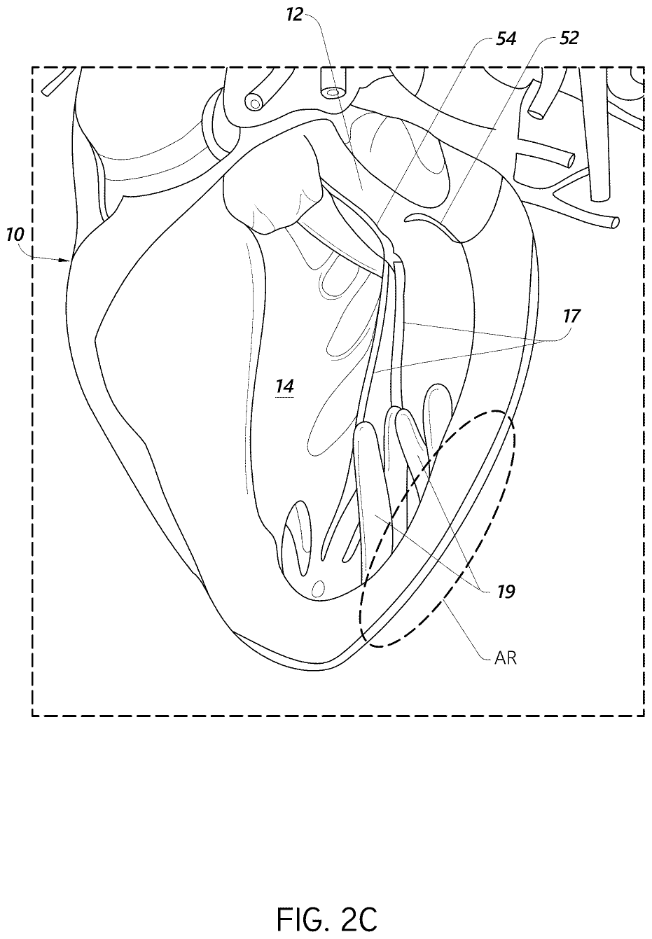

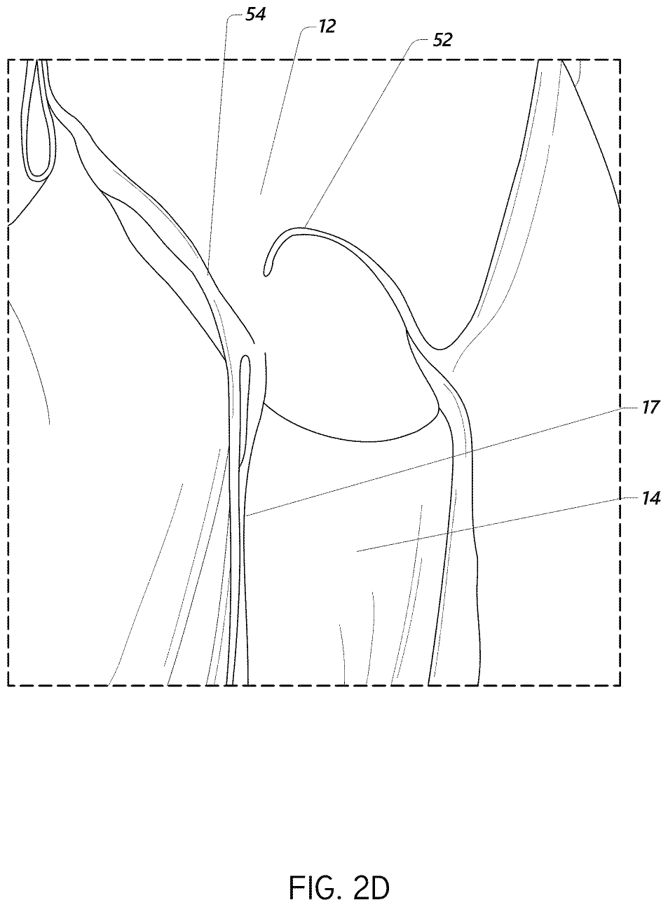

The mitral valve 22 is illustrated in FIG. 2A. The mitral valve 22 includes two leaflets, the anterior leaflet 52 and the posterior leaflet 54, and a diaphanous incomplete ring around the valve, called the annulus 53. The mitral valve 22 has two papillary muscles 19, the anteromedial and the posterolateral papillary muscles (see, e.g., FIG. 1), which attach the leaflets 52, 54 to the walls of the left ventricle 14 via the chordae tendineae 17 (see, e.g., FIG. 1).

FIG. 2B illustrates a prolapsed mitral valve 22. As can be seen with reference to FIGS. 2B-2D, prolapse occurs when a prolapsed segment of a leaflet 52, 54 of the mitral valve 22 is displaced above the plane of the mitral annulus into the left atrium 12 (see FIGS. 2C and 2D) preventing the leaflets from properly sealing together to form the natural plane or line of coaptation between the valve leaflets during systole. Because one or more of the leaflets 52, 54 malfunctions, the mitral valve 22 does not close properly, and, therefore, the leaflets 52, 54 fail to coapt. This failure to coapt causes a gap 55 between the leaflets 52, 54 that allows blood to flow back into the left atrium, during systole, while it is being ejected by the left ventricle. As set forth above, there are several different ways a leaflet may malfunction, which can thereby lead to regurgitation.

Valvular regurgitation (e.g., mitral regurgitation, tricuspid regurgitation, etc.) increases the workload on the heart and may lead to serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 22 is particularly problematic and often life threatening.

As described in detail in the '761 PCT Application and the '170 PCT Application, methods and devices are provided for performing non-invasive procedures to repair a cardiac valve, such as a mitral valve. Such procedures include procedures to repair regurgitation that occurs when the leaflets of the mitral valve do not coapt at peak contraction pressures, resulting in an undesired back flow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein and described herein to effectuate a cardiac valve repair, which will depend on the specific abnormality and the tissues involved.

After prepping and placing the subject under anesthesia, a transesophageal echocardiogram (TEE) (2D or 3D), a transthoracic echocardiogram (TTE), intracardiac echo (ICE), or cardio-optic direct visualization (e.g., via infrared vision from the tip of a 7.5 F catheter) may be performed to assess the heart and its valves.

After a minimally invasive approach is determined to be advisable, one or more incisions are made proximate to the thoracic cavity to provide a surgical field of access. The total number and length of the incisions to be made depend on the number and types of the instruments to be used as well as the procedure(s) to be performed. The incision(s) should be made in such a manner to be minimally invasive. As referred to herein, the term minimally invasive means in a manner by which an interior organ or tissue may be accessed with as little as possible damage being done to the anatomical structure through which entry is sought. Typically, a minimally invasive procedure is one that involves accessing a body cavity by a small incision of, for example, approximately 5 cm or less made in the skin of the body. The incision may be vertical, horizontal, or slightly curved. If the incision is placed along one or more ribs, it should follow the outline of the rib. The opening should extend deep enough to allow access to the thoracic cavity between the ribs or under the sternum and is preferably set close to the rib cage and/or diaphragm, dependent on the entry point chosen.

In one example method, the heart may be accessed through one or more openings made by a small incision(s) in a portion of the body proximal to the thoracic cavity, for example, between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for instance an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods further reducing the invasiveness of the procedure. See, for instance, “Full-Spectrum Cardiac Surgery Through a Minimal Incision Mini-Sternotomy (Lower Half) Technique,” Doty et al., Annals of Thoracic Surgery 1998; 65(2): 573-7 and “Transxiphoid Approach Without Median Sternotomy for the Repair of Atrial Septal Defects,” Barbero-Marcial et al., Annals of Thoracic Surgery 1998; 65(3): 771-4, the entire disclosures of each of which is incorporated herein by reference.

Once a suitable entry point has been established, the surgeon can use one or more sutures to make a series of stitches in one or more concentric circles in the myocardium at the desired location to create a “pursestring” closure. The Seldinger technique can be used to access the left ventricle in the area surrounded by the pursestring suture by puncturing the myocardium with a small sharp hollow needle (a “trocar”) with a guidewire in the lumen of the trocar. Once the ventricle has been accessed, the guidewire can be advanced, and the trocar removed. A valved-introducer with dilator extending through the lumen of the valved-introducer can be advanced over the guidewire to gain access to the left ventricle. The guidewire and dilator can be removed, and the valved-introducer will maintain hemostasis, with or without a suitable delivery device inserted therein, throughout the procedure. Alternatively, the surgeon can make a small incision in the myocardium and insert the valved-introducer into the heart via the incision. Once the valved-introducer is properly placed the pursestring suture is tightened to reduce bleeding around the shaft of the valved-introducer.

A suitable device such as a delivery device described in the '761 PCT Application and/or the '170 PCT Application, may be advanced into the body and through the valved-introducer in a manner to access the left ventricle. The advancement of the device may be performed in conjunction with sonography or direct visualization (e.g., direct transblood visualization). For example, the delivery device may be advanced in conjunction with TEE guidance or ICE to facilitate and direct the movement and proper positioning of the device for contacting the appropriate apical region of the heart. Typical procedures for use of echo guidance are set forth in Suematsu.

As shown in FIG. 3, one or more chambers, e.g., the left atrium 12, left ventricle 14, right atrium 16, or right ventricle 18 in the heart 10 may be accessed in accordance with the methods disclosed herein. Access into a chamber 12, 14, 16, 18 in the heart 10 may be made at any suitable site of entry but is preferably made in the apex region of the heart, for example, slightly above the apex 26 at the level of the papillary muscles 19 (see also FIG. 2C). Typically, access into the left ventricle 14, for instance, to perform a mitral valve repair, is gained through the process described above performed in the apical region, close to (or slightly skewed toward the left of) the median axis 28 of the heart 10. Typically, access into the right ventricle 18, for instance, to perform a tricuspid valve repair, is gained through the process described above performed in the apical region, close to or slightly skewed toward the right of the median axis 28 of the heart 10. Generally, an apex region of the heart is a bottom region of the heart that is within the left or right ventricular region and is below the mitral valve 22 and tricuspid valve 24 and toward the tip or apex 26 of the heart 10. More specifically, an apex region AR of the heart (see, e.g., FIG. 3) is within a few centimeters to the right or to the left of the septum 20 of the heart 10 at or near the level of the papillary muscles 19. Accordingly, the ventricle can be accessed directly via the apex 26, or via an off-apex location that is in the apical or apex region AR, but slightly removed from the apex 26, such as via a lateral ventricular wall, a region between the apex 26 and the base of a papillary muscle 19, or even directly at the base of a papillary muscle 19 or above. Typically, the incision made to access the appropriate ventricle of the heart is no longer than about, for example, about 0.5 cm. Alternatively, access can be obtained using the Seldinger technique described above.

The mitral valve 22 and tricuspid valve 24 can be divided into three parts: an annulus (see 53 in FIGS. 2A and 2B), leaflets (see 52, 54 in FIGS. 2A and 2B), and a sub-valvular apparatus. The sub-valvular apparatus includes the papillary muscles 19 (see FIG. 1) and the chordae tendineae 17 (see FIG. 1), which can elongate and/or rupture. If the valve is functioning properly, when closed, the free margins or edges of the leaflets come together and form a tight junction, the arc of which, in the mitral valve, is known as the line, plane or area of coaptation. Normal mitral and tricuspid valves open when the ventricles relax allowing blood from the atrium to fill the decompressed ventricle. When the ventricle contracts, chordae tendineae properly position the valve leaflets such that the increase in pressure within the ventricle causes the valve to close, thereby preventing blood from leaking into the atrium and assuring that all of the blood leaving the ventricle is ejected through the aortic valve (not shown) and pulmonic valve (not shown) into the arteries of the body. Accordingly, proper function of the valves depends on a complex interplay between the annulus, leaflets, and sub-valvular apparatus. Lesions in any of these components can cause the valve to dysfunction and thereby lead to valve regurgitation. As set forth herein, regurgitation occurs when the leaflets do not coapt properly at peak contraction pressures. As a result, an undesired back flow of blood from the ventricle into the atrium occurs.

Although the procedures described herein are with reference to repairing a cardiac mitral valve or tricuspid valve by the implantation of one or more grafts, the methods presented are readily adaptable for various types of tissue, leaflet, and annular repair procedures. In general, the methods herein are described with reference to a mitral valve 22 but should not be understood to be limited to procedures involving the mitral valve.

Repairing a cardiac valve (e.g., a mitral valve) by implanting one or more artificial cords is often influenced by a patient's particular anatomy. When the combined length of the posterior leaflet and the anterior leaflet is significantly larger than the A-P dimension of the mitral valve, the likelihood of a successful repair is significantly higher. For example, a patient having a large posterior leaflet is desirable, as a large posterior leaflet provides a large surface of coaptation with the anterior leaflet, thereby providing a sufficient seal when the leaflets coapt, e.g., to limit regurgitation. Conversely, a patient having a small posterior leaflet will have a relatively smaller surface of coaptation. Similarly, a patient having a large anterior leaflet can help lead to a desirable and successful repair. Typically, the effectiveness and durability of a repair of this nature is influenced greatly by the amount of anterior and posterior leaflet tissue coapting together during systole. Consequently, such valve repair techniques are typically less suited for patients with small anterior and/or posterior leaflets, or patients lacking tissue coaptation reserve.

The disclosed methods and devices address these and/or other issues by implanting devices in the atrium and/or ventricle, often at or near a native valve. The methods and devices can be configured to inhibit movement of a leaflet (e.g., a portion thereof) into the atrium. This can be done by treating the leaflets and/or by treating one or more natural chordae tendineae (chords). Treating the leaflets can include methods and devices that inhibit or arrest the leaflet from billowing and/or flailing into the atrium, that influence the leaflets to coapt, that take up excess tissue, and the like. Treating the chords can include methods and devices that shorten chords, that increase tension in the chords, that attach chords to the ventricle wall or to each other, and the like. In each of the disclosed methods and devices, coaptation is increased and/or valvular regurgitation is reduced. The disclosed devices and methods can be performed on a beating heart.

For each of the disclosed devices, a delivery device (e.g., a catheter) can be used to advance the device to the heart. The disclosed devices can be delivered using a percutaneous transcatheter approach such as transfemoral, transseptal, transaortic, transapical, transatrial. transradial, and the like. The disclosed devices can be crimped or otherwise configured in a delivery configuration to enable delivery to the targeted site (e.g., the atrium or ventricle). The disclosed devices can be expanded or otherwise deployed to transition from the delivery configuration to a deployed configuration. In the deployed configuration, the disclosed devices can be implanted to inhibit valvular regurgitation by inhibiting movement of a prolapsed or flailed leaflet into the atrium and/or by improving coaptation.

### Methods and Devices Directed to Affecting the Leaflets

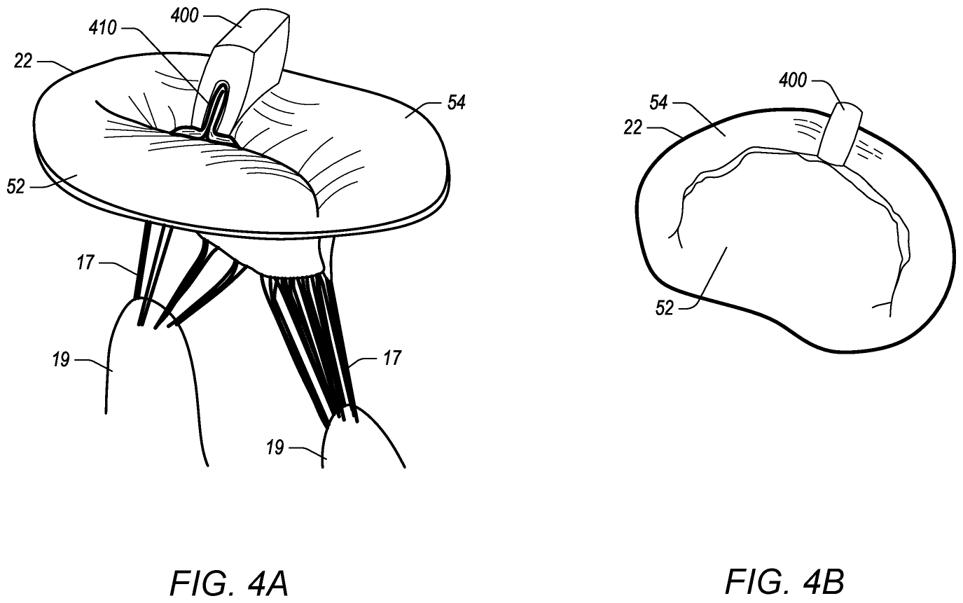

FIGS. 4A and 4B illustrate an example clip implant 400 designed to hold a portion 410 (e.g., an excess portion) of a leaflet 54. FIG. 4A illustrates a perspective view of the clip implant 400 and FIG. 4B illustrates a top view of the clip implant 400. The clip implant 400 is designed to provide an alternative to excising a portion of a leaflet (e.g., a prolapsing leaflet) and suturing the remaining portions together. In other words, the clip implant 400 is configured to secure the portion 410 of the leaflet without excising any portion of the leaflet (e.g., to secure an excess portion of a prolapsing leaflet without excising any portion of the prolapsing leaflet).

In some implementations, to do this, the clip implant 400 pulls together lateral portions of a leaflet 54 and clips the portion 410 to effectively reduce the amount of available tissue of the leaflet 54. Although the clip implant 400 is illustrated as clipping an excess portion of a posterior leaflet 54, it is to be understood that the clip implant 400 can be used to clip other portions of this or another leaflet (e.g., a portion of an anterior leaflet 52).

The clip implant 400 can be delivered via a transcatheter procedure. In some implementations, a delivery device can be maneuvered into the left atrium 12 using a transfemoral approach, which can include passing from the right atrium 16 through the septum 20 to the left atrium 12. Once in the left atrium 12, the delivery device can grab or secure a portion of the leaflet 54 near the middle of the leaflet 54. This can be done, for example, using suction, mechanical means (e.g., using a hook or barb to grab the leaflet 54), or any other suitable method. Once secured, the delivery device can gather or pull the tissue of the leaflet 54 into the atrium 12 to gather tissue of the prolapsing leaflet 54. With the excess portion 410 gathered, the clip implant 400 can be deployed from the delivery device. Once deployed, the clip implant 400 can be secured to the leaflet 54 in a way that secures the gathered portion 410 of the leaflet. The delivery device can then be withdrawn. Clipping the gathered portion 410 can reduce or eliminate valvular regurgitation and/or improve coaptation by restricting the billowing or prolapsing of the targeted leaflet 54.

The clip implant 400 can be secured near a middle portion of the targeted leaflet to pull laterally excess tissue towards the middle. Thus, the clip implant 400 can be used as an alternative to excising a middle portion of a targeted leaflet and suturing together the remaining lateral portions to remove excess tissue from the leaflet 54. In some implementations, the clip implant 400 is implanted above the annulus of the native valve 22. In some implementations, the clip implant 400 is implanted on a single leaflet. In some implementations, the clip implant 400 does not include a spacing device. A spacing device, for example, can be a device that fills a space between the leaflets 52, 54 to improve coaptation and/or to reduce valvular regurgitation.

FIG. 5 illustrates an example of magnetic implants 502, 504 configured to pull a prolapsing or billowing leaflet 52 towards the left ventricle 14. The magnetic force between the magnetic implants 502, 504 can reduce or prevent leaflet prolapse and/or other issues, thereby reducing or eliminating valvular regurgitation. Although the leaflet magnetic implant 502 is illustrated as being implanted on the anterior leaflet 52, it is to be understood that the leaflet magnetic implant 502 can be implanted on the posterior leaflet 54. Although the anchor magnetic implant 504 is illustrated as being implanted near an apex region 26 of the heart 10, it is to be understood that the anchor magnetic implant 504 can be implanted in other portions of the left ventricle 14 to pull on the leaflet magnetic implant 502 to reduce or eliminate prolapse and/or other issues.

The magnetic implants 502, 504 can be delivered via a transcatheter procedure. In some implementations, a delivery device can be maneuvered into the left ventricle 14 using a transfemoral approach, which can include passing from the right atrium 16 through the septum 20 to the left atrium 12. In some implementations, a delivery device can be maneuvered into the left ventricle 14 using a transapical approach. The delivery device can secure the anchor magnetic implant 504 in the left ventricle 14. In some implementations, the anchor magnetic implant 504 is implanted near an apex region 26 of the heart 10. The delivery device can secure the leaflet magnetic implant 502 to a targeted leaflet (e.g., the anterior leaflet 52). The leaflet magnetic implant 502 can be implanted on an atrial side of the leaflet, a ventricular side of the leaflet, the leaflet magnetic implant 502 can pierce the leaflet thus having a portion on the atrial side of the leaflet and a portion on the ventricular side of the leaflet, or the leaflet magnetic implant 502 can be clipped or secured to an edge of the leaflet. The anchor magnetic implant 504 and/or the leaflet magnetic implant 502 can be secured in place using hooks, barbs, sutures, anchors, clips, or the like. Once the magnetic implants 502, 504 are deployed, the delivery device can be withdrawn. The resulting magnetic forces from the implanted magnetic implants 502, 504 can reduce or eliminate valvular regurgitation and/or improve coaptation by restricting the billowing or prolapsing of the targeted leaflet 52. In some implementations, the anchor magnetic implant 504 is clipped to the tissue of the ventricle 14 and the magnetic fields between the magnetic implants 502, 504 serve to align the anchor magnetic implant 504 so that the magnetic fields attract the leaflet down towards the apex region 26 of the heart 10.

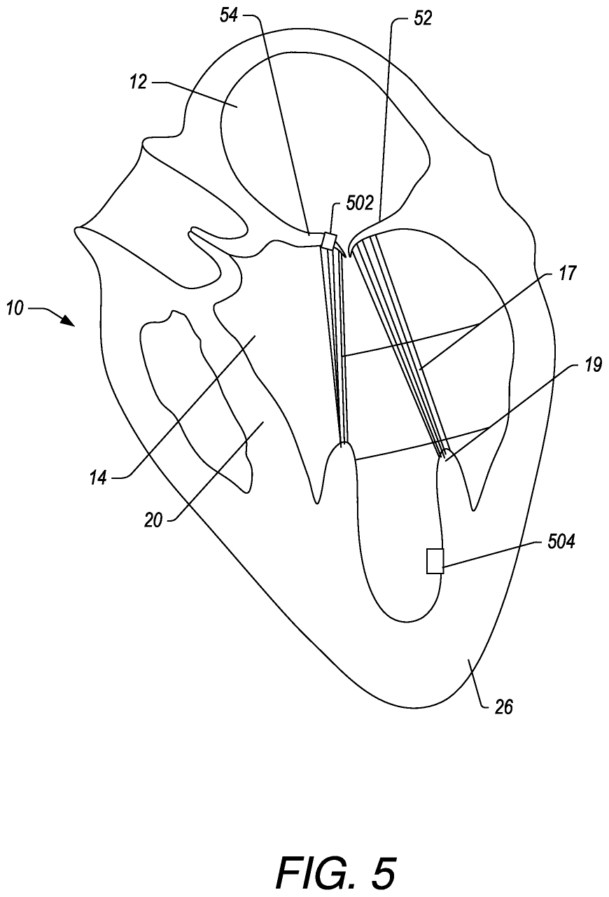

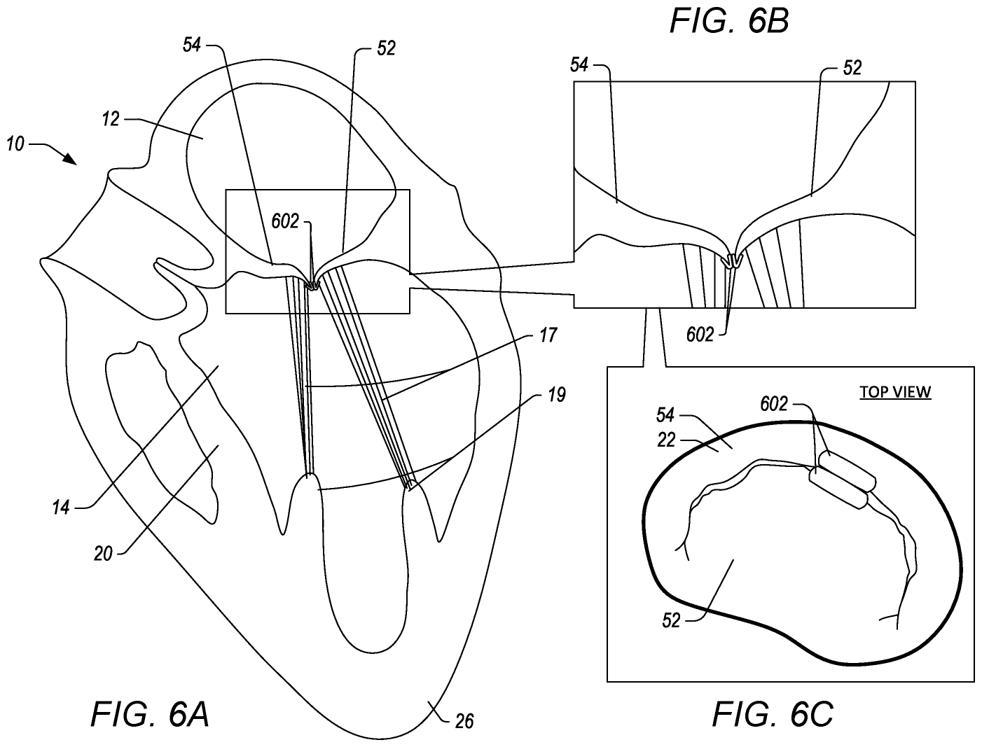

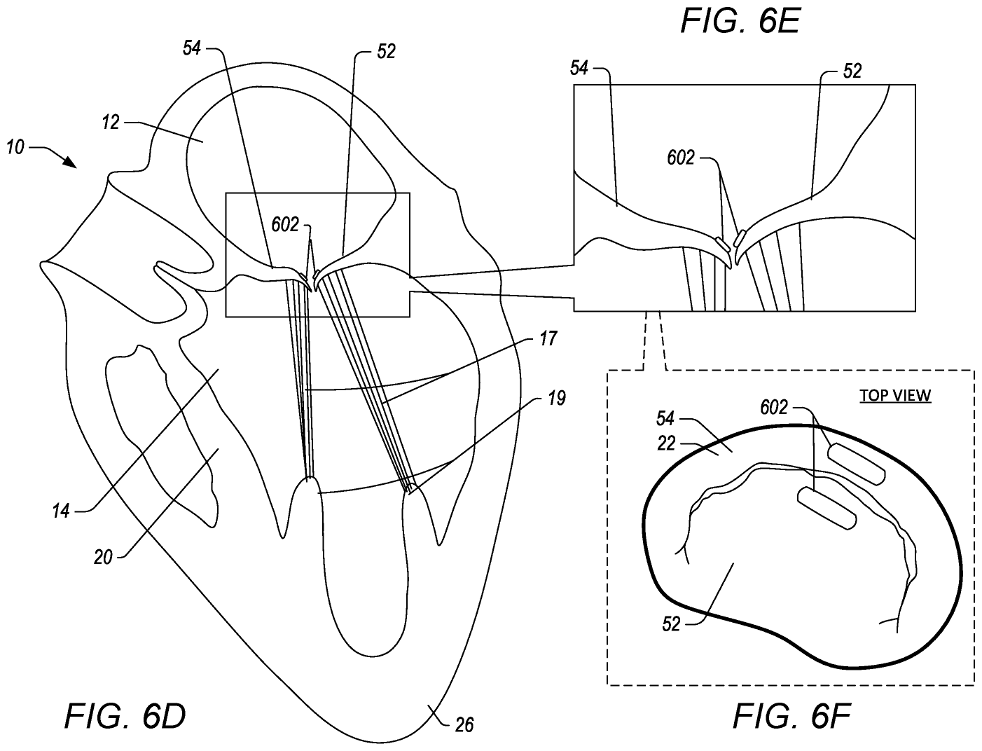

FIGS. 6A, 6B, 6C, 6D, 6E, and 6F illustrate example magnetic implants 602 configured to be clipped or secured to both leaflets 52, 54 to improve coaptation. The magnetic implants 602 can be secured to the leaflets 52, 54 so that an attractive force between the magnets influence the leaflets 52, 54 to remain closer together to improve coaptation. In some implementations, the magnetic implants 602 can be implanted on an edge of each leaflet 52, 54, as shown in FIGS. 6A, 6B, and 6C, where FIG. 6B illustrates a portion of FIG. 6A that is enlarged to show more detail of the magnetic implants 602 and FIG. 6C illustrates a top view of the magnetic implants 602 on the leaflets 52, 54. Implanting the magnetic implants 602 on the edge of each leaflet 52, 54 can be done, for example, to augment an edge of the leaflets 52, 54. In some implementations, the magnetic implants 602 can be implanted on a belly of each leaflet (e.g., higher on the leaflet into the atrium), as shown in FIGS. 6D, 6E, and 6F, where FIG. 6E illustrates a portion of FIG. 6D that is enlarged to show more detail of the magnetic implants 602 and FIG. 6F illustrates a top view of the magnetic implants 602 on the leaflets 52, 54. In some implementations, one magnetic implant 602 can be implanted on an edge of the leaflet and the other magnetic implant 602 can be implanted on a belly of the other leaflet. The location of the magnetic implants 602 can be tailored to achieve coaptation at a targeted location on the leaflets 52, 54. For example, this may take advantage of the billowing material of the prolapsing leaflet to influence coaptation so that it occurs nearer a belly of the prolapsing leaflet rather than nearer the edge of the prolapsing leaflet.

The magnetic implants 602 can be delivered via a transcatheter procedure. In some implementations, a delivery device can be maneuvered into the left atrium 12 using a transfemoral approach, which can include passing from the right atrium 16 through the septum 20 to the left atrium 12. The delivery device can secure the magnetic implants 602 to the leaflets 52, 54 either from the left atrium 12 or the left ventricle 14. The magnetic implants 602 can be secured in place using hooks, barbs, sutures, anchors, clips, or the like.

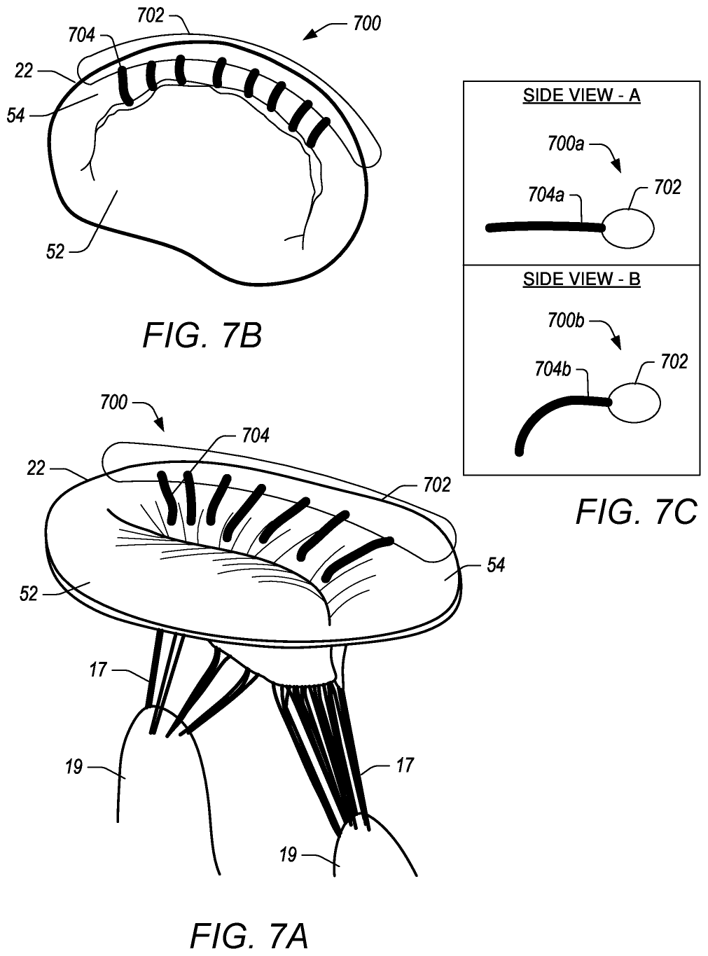

FIGS. 7A, 7B, 7C illustrate an implant 700 (shown for example as an annular implant) with a body 702 (e.g., an annular body, etc.) and hooks 704 extending from the body 702 (e.g., from an annular body). The hooks 704 are configured to extend over a portion of a leaflet 54 to reduce or prevent atrial prolapse and/or flail. The body 702 of the implant 700 can be a portion of a full annular ring (e.g., half of an annular ring, etc.). The body 702 can be configured to be limited so that it covers a portion of the native valve 22 (e.g., a portion of the native valve 22 corresponding to a leaflet or a portion of a leaflet). Thus, the implant 700 can be configured to not encircle the native valve 22. The hooks 704 can be configured to extend from the body 702 to cover a portion of the leaflet 54. The hooks 704 act to limit billowing/prolapse and/or flail of the leaflet 54. Although the implant 700 is illustrated as being implanted on the posterior leaflet 54, it is to be understood that the implant 700 can be implanted on the anterior leaflet 52. In some implementations, the body 702 is implanted on or near the annulus of the native valve 22.

The implant 700 can be delivered via a transcatheter procedure. In some implementations, a delivery device can be maneuvered into the left atrium 12 using a transfemoral approach, which can include passing from the right atrium 16 through the septum 20 to the left atrium 12. The delivery device can secure the annular implant 700 to the leaflets 54. The annular implant 700 can be secured in place using hooks, barbs, sutures, anchors, clips, or the like. To deploy the annular implant 700, the annular implant 700 can be in a delivery configuration with the hooks 704 positioned toward the delivery device so that the hooks 704 do not scrape against the lining of the delivery device as they are deployed. In some implementations, deployment of the annular implant 700 includes withdrawing the body 702 from the delivery device in such a way that each hook 704 exits the delivery device separately.

In some implementations, the hooks 704 can be evenly spaced along the body 702 (e.g., along the annular body, etc.). In some implementations, a majority of the hooks 704 extend from a middle portion of the body 702 so that the majority of the plurality of hooks are concentrated in the middle portion of the body 702. The hooks 704 can be made of any suitable material, such as Nitinol or polymer material. The hooks 704 can be configured to be sufficiently strong to prevent the leaflet 54 from prolapsing. In some implementations, as illustrated in the top portion of FIG. 7C, an annular implant 700a can have hooks 704a that are relatively straight as they extend from the annular body 702. In some implementations, as illustrated in the bottom portion of FIG. 7C, an annular implant 700b can have hooks 704b that curve downwards toward the ventricle.

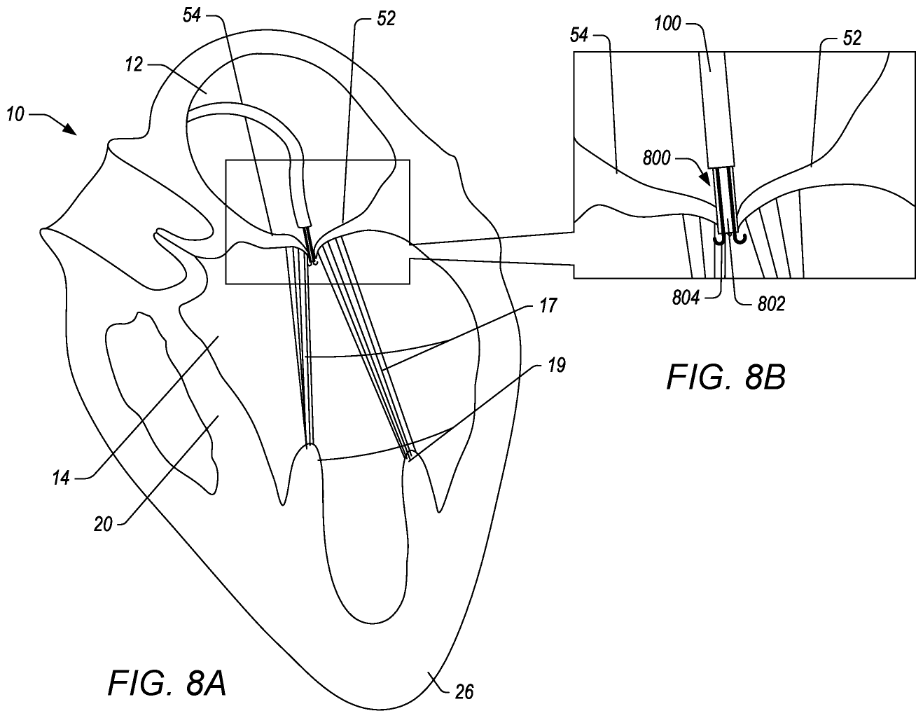

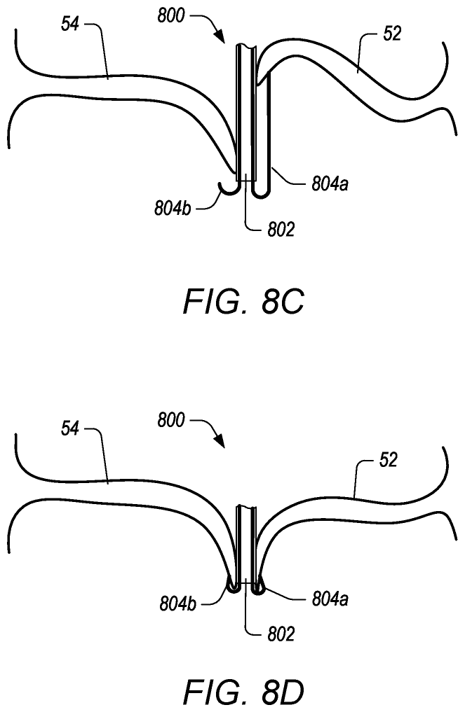

FIGS. 8A, 8B, 8C, and 8D illustrate implantation of an example implant or leaflet clipping implant 800. The leaflet clipping implant 800 includes a spacing material 802 and paddles 804 with lengths that are independently adjustable. This allows clipping of the prolapsing leaflet followed by clipping of the non-prolapsing leaflet with reduced stress on the implant 800 and/or reduced stress in the engagement of the leaflet. The paddles 804 can be configured to secure to a prolapsing leaflet, pull the prolapsing leaflet towards the non-prolapsing leaflet, secure to the non-prolapsing leaflet, and secure both leaflets to the spacing material 802 between the leaflets to reduce or eliminate leaflet prolapse, valvular regurgitation, and/or other issues. The paddles 804 can be coupled to the spacing material 802. The paddles 804 can extend from the spacing material 802. In some implementations, the paddles 804 are pliable so that they can be coupled to the spacing material 802 along a portion of the spacing material 802 and also extend from the spacing material 802 to contact and secure portions of the leaflets 52, 54 to prevent or reduce leaflet prolapse and/or flail. The paddles 804 each include securing mechanisms that are configured to secure, grab, or clip a portion of the leaflets 52, 54 to be able to draw the leaflets toward the spacing material 802. The securing mechanisms can include, for example and without limitation, hooks, anchors, clips, magnets, clamps, screws, staples, sutures, needles, and the like or any combination of two or more of these mechanisms.

The leaflet clipping implant 800 can be delivered via a transcatheter approach. In some implementations, a delivery device 100 can be maneuvered into the left atrium 12 using a transfemoral approach, which can include passing from the right atrium 16 through the septum 20 to the left atrium 12. The delivery device 100 can position the leaflet clipping implant 800 between the leaflets 52, 54, as shown in FIGS. 8A and 8B. The leaflet clipping implant 800 extends from a distal end of the delivery device 100. With the leaflet clipping implant 800 between the leaflets, the paddles 804 can be maneuvered to attach to the leaflets 52, 54. The paddles 804 can be configured to secure the leaflets 52, 54 to the spacing material 802. The spacing material 802 is configured to be implanted between the leaflets 52, 54 while the paddles 804 are configured to secure central portions of the leaflets 52, 54 to the spacing material 802 to reduce or prevent valvular regurgitation. The paddles 804 can include hooks, barbs, sutures, anchors, clips, or the like to secure the paddles 804 to the leaflet tissue.

An example method for deploying the leaflet clipping implant 800 is illustrated in FIGS. 8C and 8D, which occur after delivery of the leaflet clipping implant 800 as shown in FIGS. 8A and 8B. To deploy the leaflet clipping implant 800, a first paddle 804a is extended to attach to the prolapsing leaflet (e.g., the anterior leaflet 52), as shown in FIG. 8C. Manipulation of the paddles 804a, 804b can be accomplished at a proximal end of the delivery device 100. Once secured to the prolapsing leaflet 52, the paddle 804a is retracted to pull the prolapsing leaflet 52 towards the non-prolapsing leaflet 54, as shown in FIG. 8D. Once the prolapsing leaflet 52 is positioned near the non-prolapsing leaflet 54, the paddle 804b can be secured to the non-prolapsing leaflet, as shown in FIG. 8D. With the paddles 804a, 804b secured to the leaflets 52, 54, the paddles can be cut to length and secured to the spacing material 802. In this deployed configuration, the delivery device 100 can be removed, leaving the spacing material 802 between the leaflets 52, 54, with the paddles 804a, 804b securing the leaflets 52, 54 to the spacing material 802. The paddles 804a, 804b can be made of any suitable material including Nitinol. In some implementations, in the deployed configuration, edges of the leaflets 52, 54 are positioned within a hooking portion of the paddles 804a, 804b, as shown in FIG. 8D.

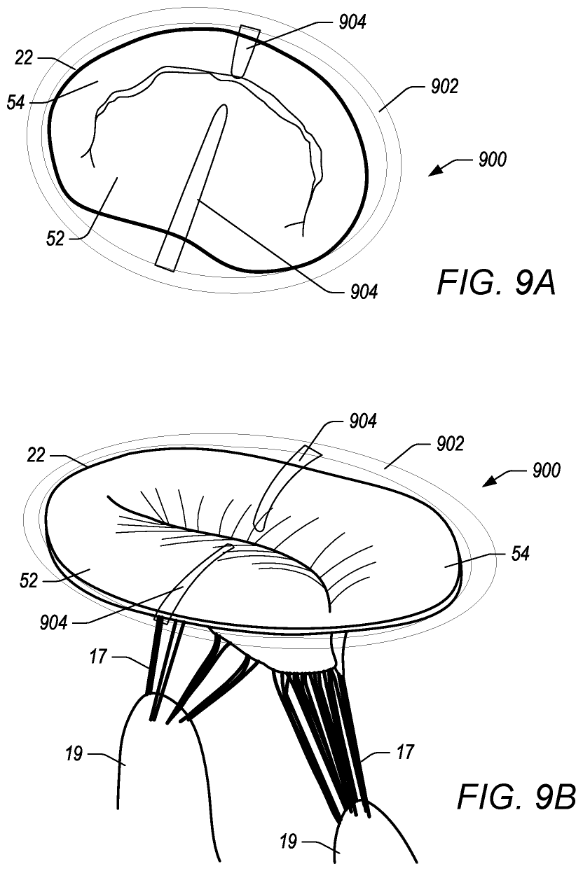

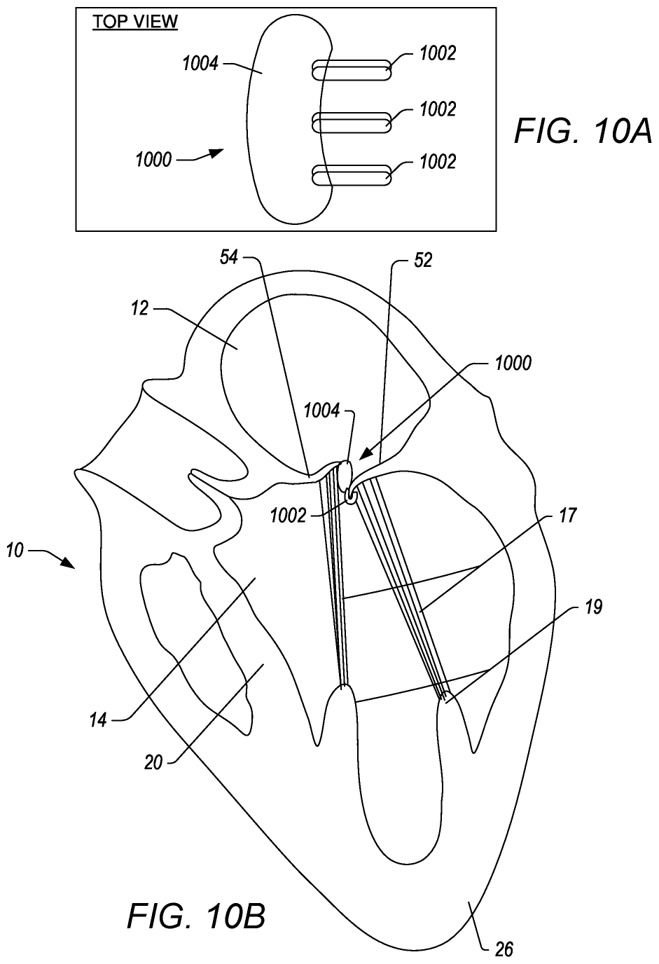

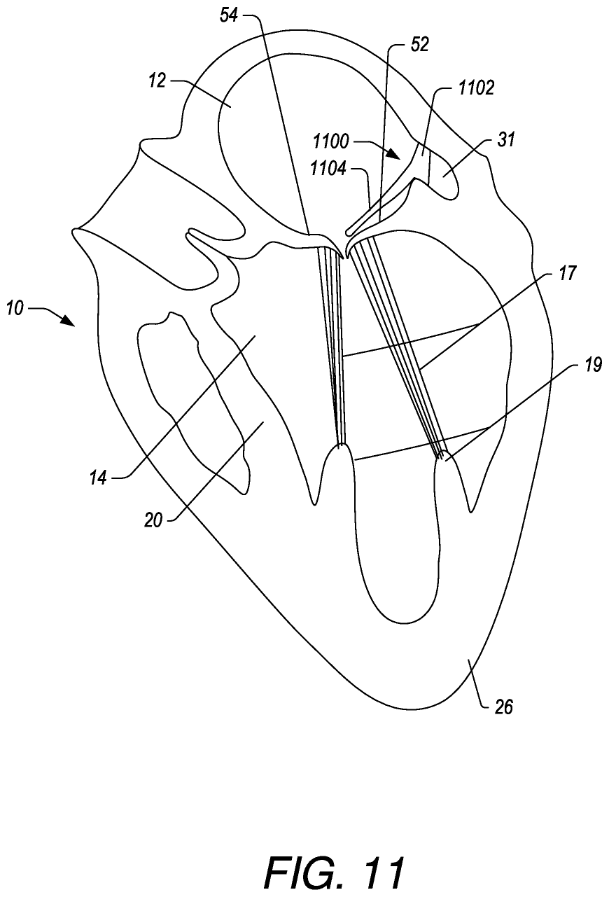

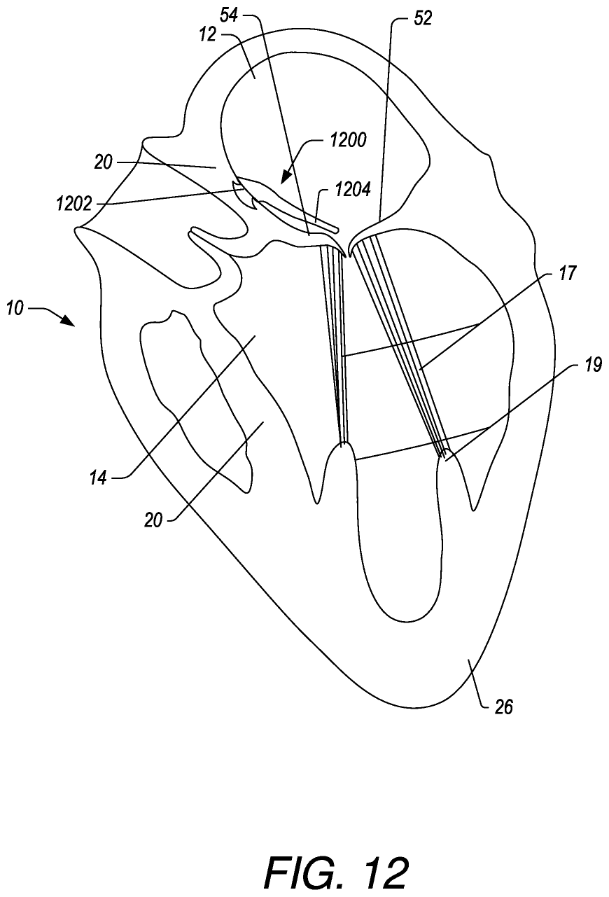

FIGS. 9A and 9B illustrate a top view and a perspective view, respectively, of an example flanged annular implant 900 with an annular body 902 and flanges 904 extending from the annular body 902. The flanges 904 are configured to extend over a portion of both leaflets 52, 54 to reduce or prevent atrial prolapse and/or flail. The annular body 902 of the flanged annular implant 900 can be a full annular ring. The annular body 902 can be configured to encircle the native valve 22. The flanges 904 can be configured to extend from the annular body 902 to cover a portion of each leaflet 52, 54. The flanges 904 act to limit prolapse and/or flail of a leaflet.

The flanged annular implant 900 can be delivered via a transcatheter procedure. In some implementations, a delivery device can be maneuvered into the left atrium 12 using a transfemoral approach, which can include passing from the right atrium 16 through the septum 20 to the left atrium 12. The delivery device can secure the flanged annular implant 900 to the native valve 22 (e.g., to the annulus 53). The flanged annular implant 900 can be secured in place using hooks, barbs, sutures, anchors, clips, or the like. To deploy the flanged annular implant 900, the annular body 902 of the flanged annular implant 900 can be secured to the native valve 22 with the flanges 904 retracted. Wires can be advanced using the delivery device, wherein the wires are configured to extend the flanges 904 from the annular body 902 of the flanged annular implant 900. The flanged annular implant 900 can include a cloth material that has some elasticity. By advancing the wires, the flanges 904 form from the cloth surrounding the annular body 902 to extend over the leaflets 52, 54. The wires within the flanges 904 can be shape set material, such as Nitinol. In some implementations, the annular body 902 is inflatable (e.g., using a fluid such as saline) and deploying the flanged annular implant 900 comprises inflating the annular body 902 which in turn causes the flanges 904 to extend inward away from the annular body 902 and over the leaflets 52, 54. In such implementations, the flanges 904 comprise pliable material that can be inflated by the inflating fluid (e.g., saline). Although two flanges 904 are shown here, it is to be understood that 2 or more flanges 904 can be configured to extend from the annular body 902 of the flanged annular implant 900. The flanges 904 can be configured to be sufficiently strong to prevent the leaflets 52, 54 from prolapsing. The flanges 904 can be configured to extend inward and downward (toward the ventricle) from the annular body 902. The flanges 904 are configured to apply a downward force on the leaflets 52, 54 to limit or prevent leaflet prolapse and/or flail. The extent of the flanges 904 from the annular body 902 can be configured and, in some implementations, each flange 904 can be adjusted independently. The annular body 902 is implanted on the atrial side of the native valve 22. In some implementations, the flanges 904 can be extended and filled with a foam material or a hardening material to finish implantation.

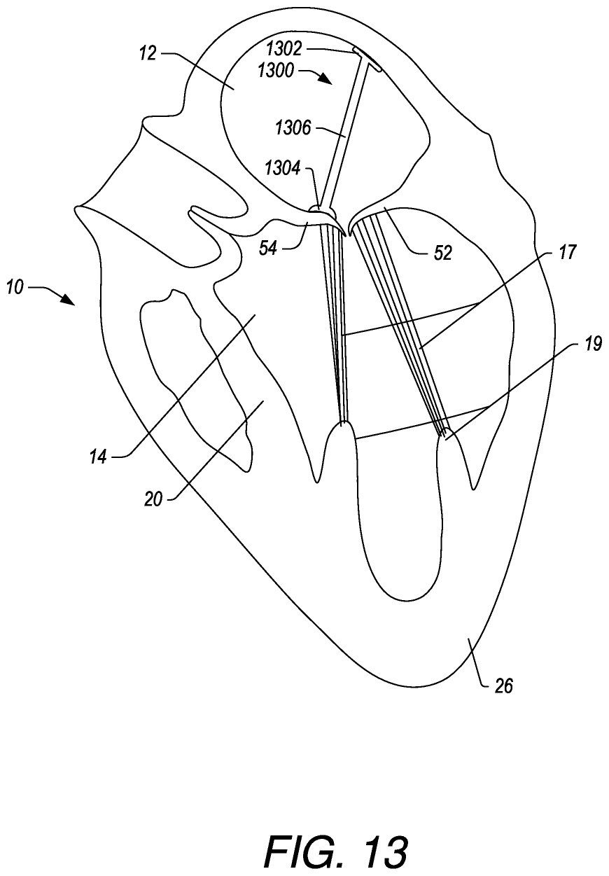

FIGS. 10A and 10B illustrate a gap-filling implant 1000 configured to secure to an edge of a non-prolapsing leaflet and to provide spacing material for coaptation with the prolapsing leaflet. The gap-filling implant 1000 includes spacing material 1004 and one or more clips 1002 to attach to a free edge of the non-prolapsing leaflet 52, the spacing material 1004 configured to fill the gap between the prolapsing leaflet 54 and the non-prolapsing leaflet 52.

The gap-filling implant 1000 can be delivered via a transcatheter procedure. In some implementations, a delivery device can be maneuvered into the left atrium 12 using a transfemoral approach, which can include passing from the right atrium 16 through the septum 20 to the left atrium 12. The delivery device can position the gap-filling implant 1000 in the left atrium 12 between the leaflets 52, 54. Once in position, the delivery device can deploy the one or more clips 1002 to attach to the free edge of the non-prolapsing leaflet 52. In some implementations, the gap-filling implant 1000 is configured to fill in approximately the entire length of the free edge of the leaflet 52. Thus, the number and design of the clips 1002 can be configured to achieve this aim. For example, 3 or more clips can be used to secure the gap-filling implant 1000 along the edge of the leaflet 52. As another example, a clip 1002 can be positioned near a center of the edge of the leaflet 52 and the clip 1002 can be configured to be sufficiently wide so that the spacing material 1004 can fill in the gap between the leaflets 52, 54. In some implementations, the spacing material 1004 includes a cloth with a coiled shape set material within the cloth. In some implementations, the spacing material 1004 is inflatable using a fluid such as saline. The spacing material 1004 can be curved to follow a natural curvature of the leaflets 52, 54.

FIG. 11 illustrates an LAA implant 1100 configured to be anchored in a left atrial appendage 31 (LAA) and to protrude over the anterior leaflet 52 to inhibit or prevent the anterior leaflet 52 from prolapsing. The LAA implant 1100 includes an anchor 1102 and a protruding flange 1104, the protruding flange 1104 configured to inhibit prolapsing of the anterior leaflet 52 when the anchor 1102 is anchored in the LAA 31.

The LAA implant 1100 can be delivered via a transcatheter procedure. In some implementations, a delivery device can be maneuvered into the left atrium 12 using a transfemoral approach, which can include passing from the right atrium 16 through the septum 20 to the left atrium 12. In the left atrium, the delivery device can anchor the LAA implant 1100 in the LAA 31 by securing the anchor 1102 in the ostium or other portion of the LAA 31. The anchor 1102 can include, for example, coils, hooks, barbs, or the like to secure the LAA implant 1100 to the LAA 31. In some implementations, the anchor 1102 is configured to allow fluid to pass in and out of the LAA 31. In some implementations, the anchor 1102 can act as an LAA occluder, preventing fluid flow into the LAA to inhibit or prevent blood clots from forming in the LAA 31. The protruding flange 1104 can extend from the anchor 1102 and can provide a downward force (from the atrium toward the ventricle) to inhibit or prevent the anterior leaflet 52 from prolapsing. In some implementations, the protruding flange 1104 is configured to lie along a portion of the anterior leaflet 52 to restrain movement of the leaflet into the left atrium 12. The protruding flange 1104 can be made of a mesh material and can include shape set metals (e.g., Nitinol) and/or it can be inflatable (e.g., using a fluid such as saline).

FIG. 12 illustrates a septal implant 1200 configured to be anchored in the septum 20 between the left atrium 12 and the right atrium 16 and to protrude over the posterior leaflet 54 to inhibit or prevent the posterior leaflet 54 from prolapsing. The septal implant 1200 includes an anchor 1202 and a protruding flange 1204, the protruding flange 1204 configured to inhibit prolapsing of the posterior leaflet 54 when the anchor 1202 is anchored in the septum 20.

The septal implant 1200 can be delivered via a transcatheter procedure. In some implementations, a delivery device can be maneuvered into the left atrium 12 using a transfemoral approach, which can include passing from the right atrium 16 through the septum 20 to the left atrium 12. In the left atrium, the delivery device can anchor the septal implant 1200 in the septum 20 by securing the anchor 1202 in the atrium wall. In some implementations, the septal implant 1200 can be implanted in the hole in the septum 20 created by the delivery device. The anchor 1202 can include, for example, coils, hooks, barbs, or the like to secure the septal implant 1200 to the septum 20. The protruding flange 1204 can extend from the anchor 1202 and can provide a downward force (from the atrium toward the ventricle) to inhibit or prevent the posterior leaflet 54 from prolapsing. In some implementations, the protruding flange 1204 is configured to lie along a portion of the posterior leaflet 54 to restrain movement of the leaflet into the left atrium 12. The protruding flange 1204 can be made of a mesh material and can include shape set metals (e.g., Nitinol) and/or it can be inflatable (e.g., using a fluid such as saline).

In some implementations, the LAA implant 1100 and the septal implant 1200 can be combined to treat prolapsing leaflets. In this way, both the anterior leaflet 52 and the posterior leaflet 54 can be inhibited from moving into the left atrium 12.

FIG. 13 illustrates an atrial compression implant 1300 configured to be anchored in the wall of the left atrium 12 and anchored on or secured to a prolapsing leaflet to inhibit or prevent prolapsing of the leaflet. The atrial compression implant 1300 includes an atrial anchor 1302, a leaflet anchor 1304, and a shaft 1306 connecting the atrial anchor 1302 and the leaflet anchor, the shaft 1306 configured to inhibit or prevent leaflet prolapse and/or flail (e.g., by providing a resistance to upward forces and/or by providing a downward force). The atrial anchor 1302 can be embedded or anchored to the atrial wall. The leaflet anchor 1304 can be embedded or anchored to the leaflet 54. In some implementations, the shaft 1306 includes a compressive component (e.g., a coil or spring) to provide elastic resistance to the leaflet 54 during normal operation of the heart 10. In some implementations, the shaft 1306 allows the leaflet 54 to move downward into the ventricle 14 but resists upward movement into the atrium 12. For example, a stiff rod can be encased in an elastic cloth or material that is anchored to the leaflet 54. When the leaflet 54 moves downward, the elastic material allows the movement, and when the leaflet 54 moves upward, the rigid or stiff rod provides resistance at a certain point to inhibit further upward movement. Although the atrial compression implant 1300 is illustrated as being implanted on the posterior leaflet 54, it is to be understood that the atrial compression implant 1300 can be implanted on the anterior leaflet 52.

The atrial compression implant 1300 can be delivered via a transcatheter procedure. In some implementations, a delivery device can be maneuvered into the left atrium 12 using a transfemoral approach, which can include passing from the right atrium 16 through the septum 20 to the left atrium 12. In the left atrium, the delivery device can anchor the atrial compression implant 1300 to the atrial wall above the prolapsing leaflet and to the prolapsing leaflet. The atrial anchor 1302 and/or the leaflet anchor 1304 can include, for example, coils, hooks, barbs, or the like to secure the anchors 1302, 1304 to the atrial wall and to the leaflet 54, respectively. The shaft 1306 extends from the atrial anchor 1302 to the leaflet anchor 1304 and provides a downward force (from the atrium toward the ventricle) to inhibit or prevent the posterior leaflet 54 from prolapsing. In some implementations, the shaft 1306 is configured to have compression characteristics that do not adversely affect the atrial wall during operation of the heart 10.

In some implementations, the atrial anchor 1302 includes a stent that deploys into the roof of the atrium 12. In some implementations, the atrial compression implant 1300 is implanted in such a way that the shaft 1306 is angled to improve the force vector. For example, to inhibit the posterior leaflet 54 from prolapsing, the atrial anchor 1302 can be implanted directly above the anterior leaflet 52. The resulting angle of the shaft 1306 advantageously provides a more perpendicular force on the posterior leaflet 54, which may be advantageous. As another example, to inhibit the anterior leaflet 52 from prolapsing, the atrial anchor 1302 can be implanted directly above the posterior leaflet 54. The resulting angle of the shaft 1306 advantageously provides a more perpendicular force on the anterior leaflet 52. The angle of the shaft 1306 relative to the prolapsing leaflet at a point where the leaflet anchor 1304 is anchored to the prolapsing leaflet is approximately perpendicular when the native valve 22 is closed. In some implementations, the atrial compression implant 1300 can be used in conjunction with the LAA implant 1100 and/or the septal implant 1200.

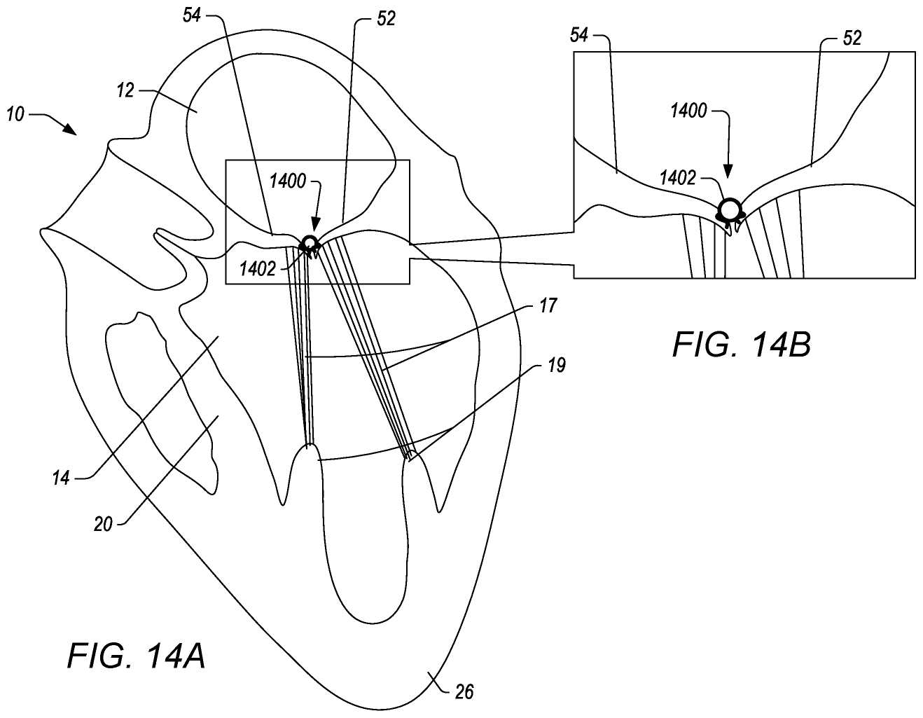

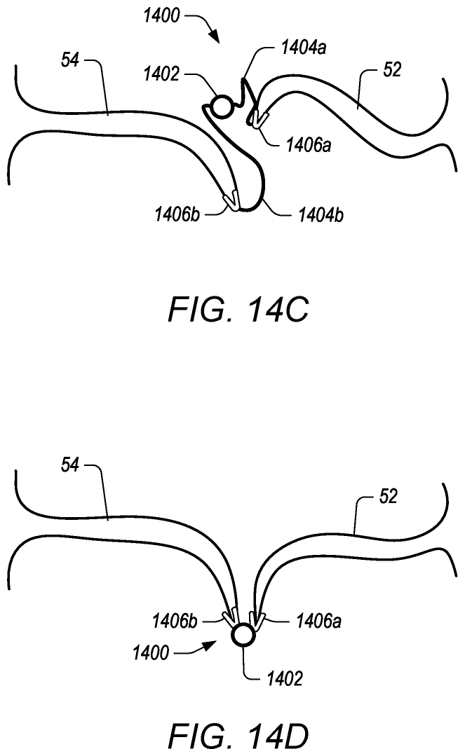

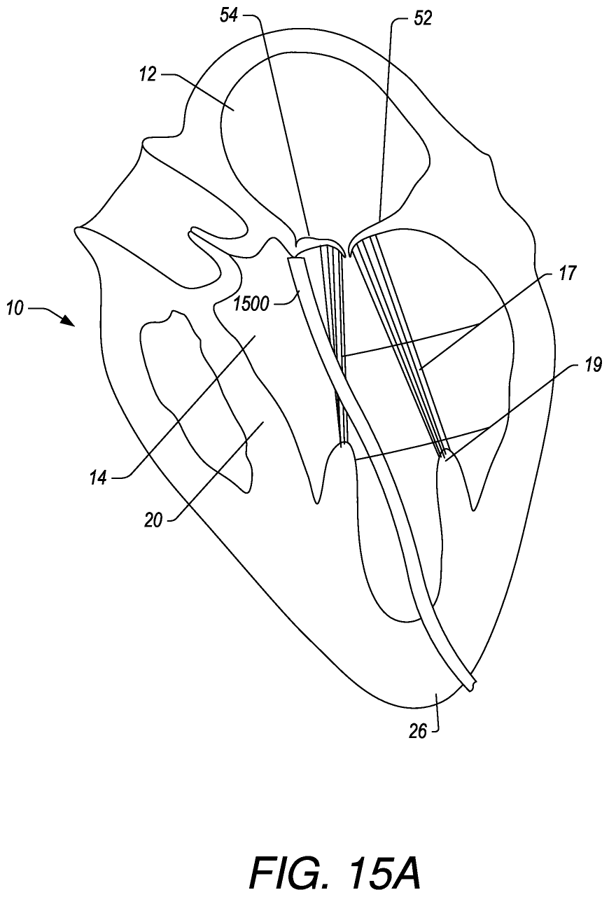

FIGS. 14A, 14B, 14C, and 14D illustrate a cinching leaflet implant 1400 configured to attach to the two leaflets 52, 54 and to pull the leaflets 52, 54 toward one another. The cinching leaflet implant 1400 includes two or more free-edge clipping implants 1406a, 1406b that are joined by sutures 1404a, 1404b and a cinching mechanism 1402. In instances where the leaflets 52, 54 are naturally separated by a relatively large distance due to chord elongation and/or leaflet prolapse, the cinching leaflet implant 1400 can be used to join the two leaflets 52, 54 together to inhibit or prevent valvular regurgitation. This may be advantageous where typical systems and devices fail due to the large separation. The free-edge clipping implants 1406a, 1406b can be used to clip to leaflets 52, 54 that are separated by a relatively large distance. The sutures 1404a, 1404b and the cinching mechanism can then be used to pull the leaflets together using the free-edge clipping implants 1406a, 1406b. The cinching mechanism 1402 can be configured to spool the sutures that connect the cinching mechanism 1402 to the free-edge clipping implants 1406a, 1406b. In some implementations, the cinching mechanism 1402 includes a spooling mechanism that includes, without limitation, a wheel, bearing, and/or a spool that is configured to rotate to spool the sutures 1404a, 1404b within or around the spooling mechanism. The cinching mechanism 1402 can include a locking component (e.g., a rod, spring, disk, or the like) configured to lock the free-edge clipping implants 1406a, 1406b and/or the sutures 1404a, 1404b in place. The locking component can interact with the spooling mechanism to allow or inhibit spooling to both lengthen and shorten the sutures 1404a, 1404b.

An example method of use is illustrated in FIGS. 14C and 14D. The cinching leaflet implant 1400 can be delivered via a transcatheter procedure. In some implementations, a delivery device can be maneuvered into the left atrium 12 using a transfemoral approach, which can include passing from the right atrium 16 through the septum 20 to the left atrium 12. In the left atrium, the delivery device can attach a first free-edge clipping implant 1406a to a first leaflet edge (e.g., the anterior leaflet 52). Next, the delivery device can attach a second free-edge clipping implant 1406b to a second leaflet edge (e.g., the posterior leaflet 54), as shown in FIG. 14C. The delivery device can then be used to operate the cinching mechanism 1402 to pull the leaflets 52, 54 together via the sutures 1404a, 1404b, as shown in FIG. 14D. The delivery device can then be withdrawn.

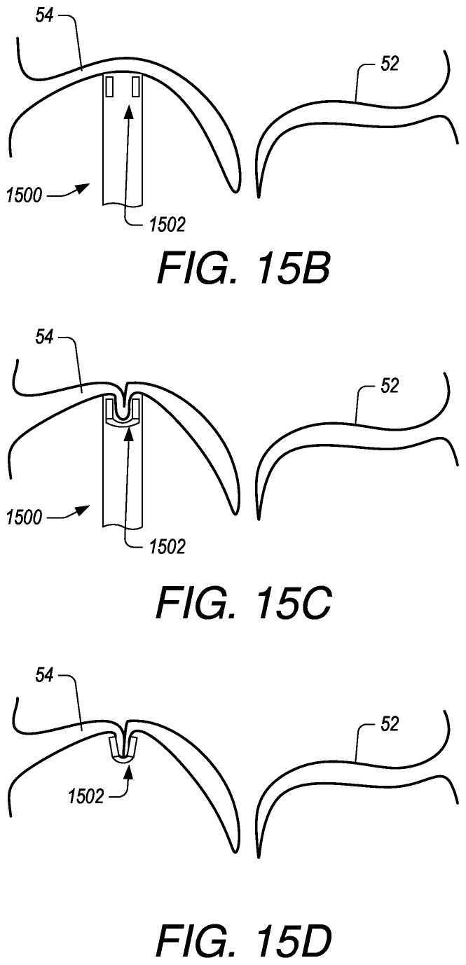

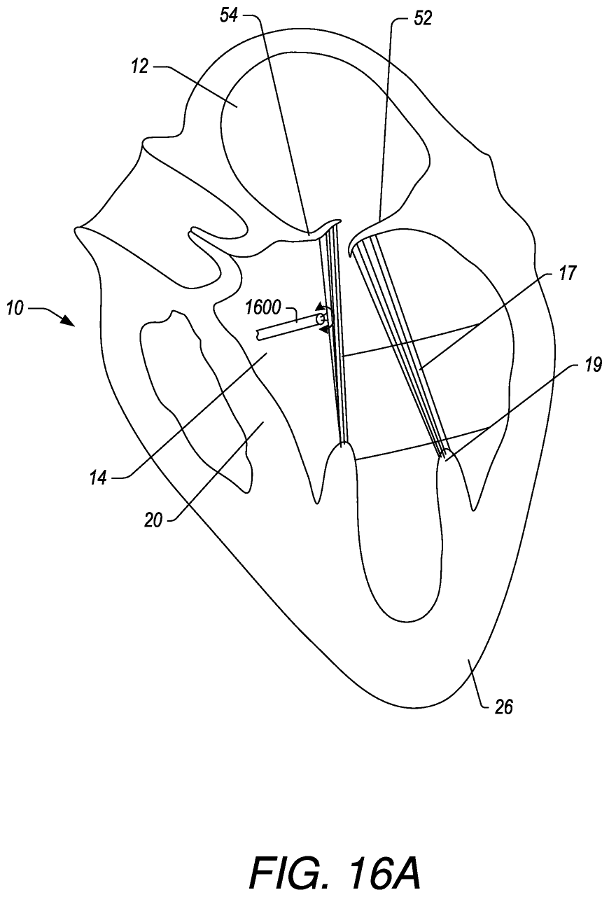

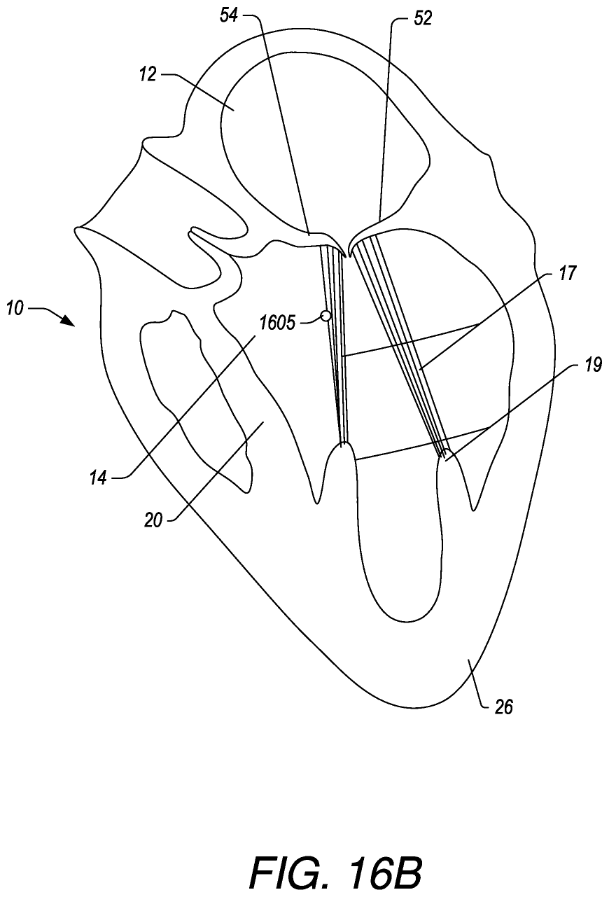

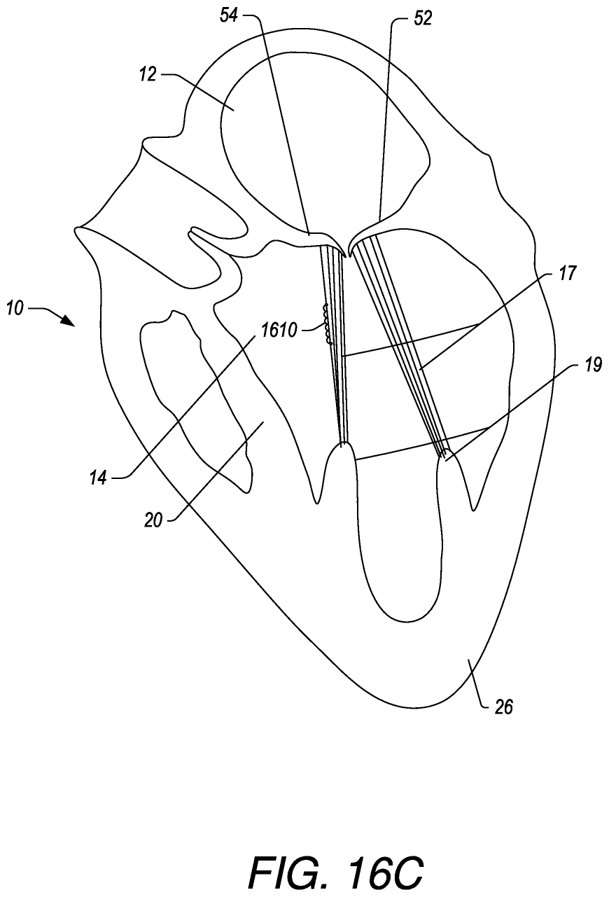

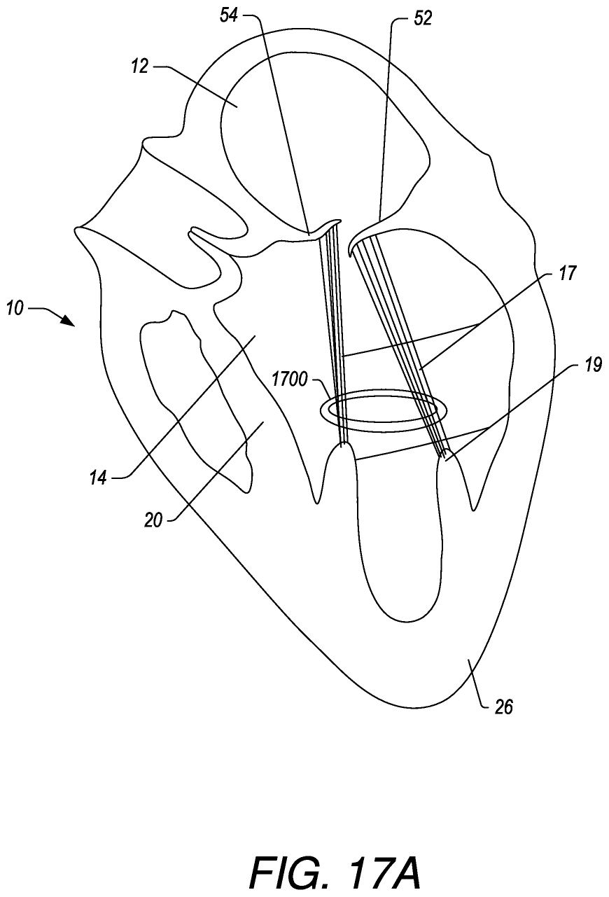

FIGS. 15A-15D illustrate an example method for excising and clipping together leaflets to treat a leaflet, e.g., to reduce or prevent leaflet prolapse. As shown in FIG. 15A, a delivery device 1500 is delivered into the left ventricle 14. As illustrated, this can be accomplished using a transapical approach, but other approaches may also be used. The delivery device 1500 is advanced to an underside of the targeted leaflet (e.g., the posterior leaflet 54), as shown in FIG. 15B. The delivery device 1500 uses suction or mechanical means to draw a billowing portion of the leaflet 54 into the delivery device 1500, as shown in FIG. 15C. Within the delivery device 1500, cauterization can be used to excise an excess portion of the leaflet 54. A clip 1502 can then be used to clip together the cauterized portion of the leaflet 54 to reduce or eliminate leaflet prolapse and/or another issue. The clip 1502 can be similar to the clip implant 400 described herein with reference to FIG. 4.

### Methods and Devices Directed to Affecting Chordae

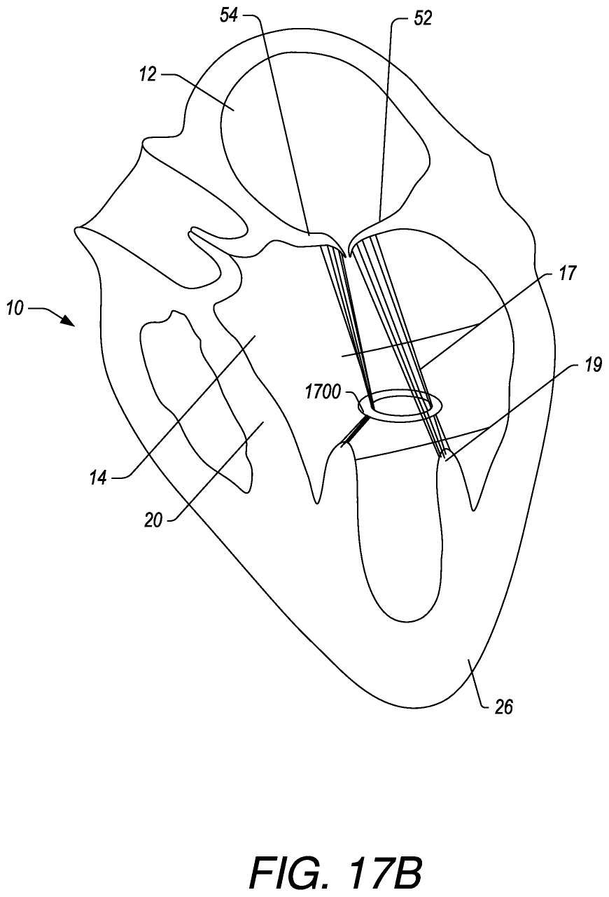

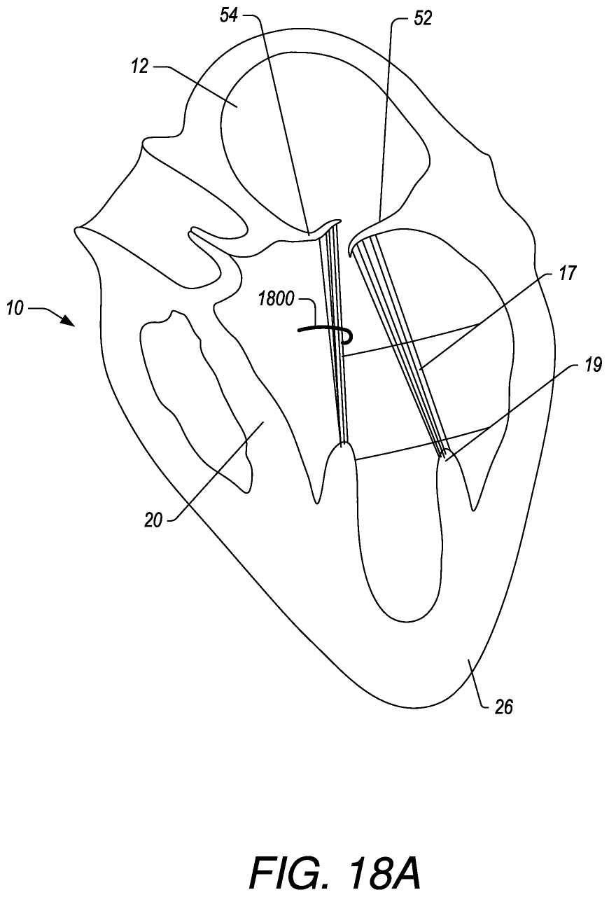

FIGS. 16A-16C illustrate an example device 1600 and associated method for reducing chord length. In some implementations, the device 1600 spools an elongated chord around a chordal implant. In some implementations, the device 1600 implants a spring implant on an elongated chord to shorten the chord.

The device 1600 includes a twisting component that can twist a selected or targeted chord to shorten the chord. The device 1600 can be delivered into the left ventricle 14 using a transapical approach or a transfemoral approach, for example. Once in the left ventricle 14, the device 1600 twists or spools a targeted chord, as shown in FIG. 16A. With the targeted chord twisted or spooled, a spooling or chordal implant 1605 (e.g., a clip) can be used to secure the twisted chord to secure it in a shortened configuration, as shown in FIG. 16B. Alternatively or additionally, a spring or elastic implant 1610 can be attached above and below the twisted portion to pull end portions of the targeted chord toward one another to secure it in the shortened configuration, as shown in FIG. 16C.

In some implementations, the delivery device includes a twisting component that can grab or temporarily secure a portion of a targeted chord for twisting or spooling. In some implementations, the delivery device can deploy a spooling mechanism (e.g., the spooling implant 1605) that can attach to the chord. Once attached, the spooling mechanism can be operated to twist or spool the chord to which it is attached. In addition, the spooling mechanism can be locked to secure the chord in a shortened configuration. In some implementations, twisting the targeted chord about once or twice may be sufficient to achieve a targeted shortening of the chord to reduce or prevent valvular regurgitation due to elongated chords. In some implementations, the spring implant 1610 includes clamps or crimps on either side of the spring implant 1610 to secure the spring implant 1610 to the targeted chord. In some implementations, the spooling implant 1605 and/or the spring implant 1610 is configured to shorten a single chord at a time. In some implementations, the spring implant 1610 includes one end anchored to the leaflet insertion point or to the papillary muscle 19.

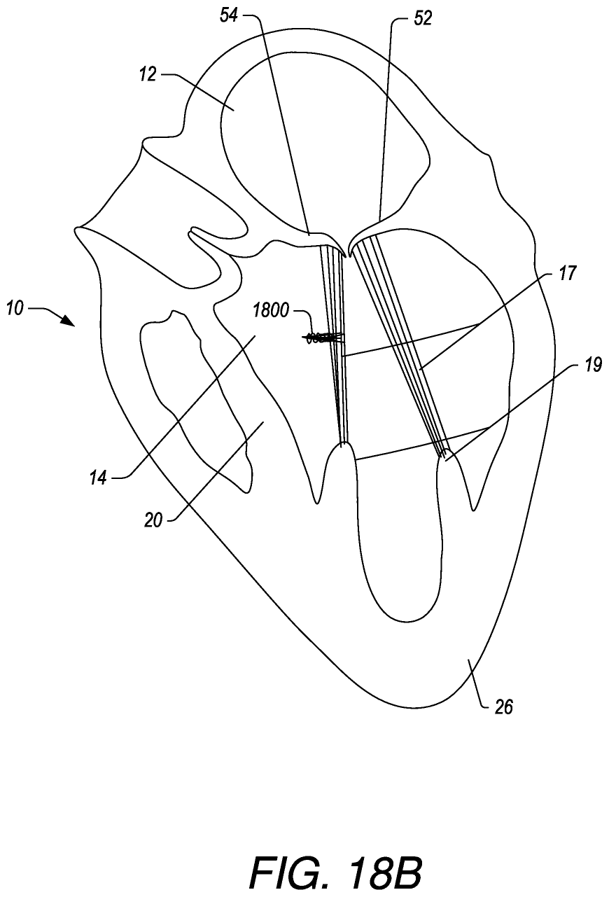

FIGS. 17A and 17B illustrate a chordal ring implant 1700 configured to bundle elongated chords with normal chords. The chordal ring implant 1700 is configured to cinch the elongated chords to the normal chords to reduce the length of the elongated chords. Reduction of the length of elongated chords can reduce or prevent leaflet prolapse, valvular regurgitation, and/or other issues.

The chordal ring implant 1700 can be delivered to the left ventricle 14 via a transcatheter procedure. For example, a transapical approach can be used to deliver the chordal ring implant 1700 to the left ventricle 14. In the left ventricle 14, the delivery device can wrap the chordal ring implant 1700 around elongated chords and normal chords, as shown in FIG. 17A. In this way, the chordal ring implant 1700 pulls the elongated chords to the normal chords to effectively shorten the elongated chords. To cinch the chordal ring implant 1700, a suture or wire that wraps around the chordal ring implant 1700 can run to a proximal portion of the delivery device. Actuation of the suture or wire can pull the chordal ring implant 1700 to cinch the ring around the chords, as shown in FIG. 17B. The suture or wire can be wound around a circumference of the chordal ring implant 1700. The chordal ring implant 1700 can include a cloth covering or PTFE tubing. In some implementations, the chordal ring implant 1700 does not include anchors. The chordal ring implant 1700 in a delivery configuration is a disconnected ring. To transition to the deployment configuration, the chordal ring implant 1700 can be fed around the chords 17. The ends of the chordal ring implant 1700 can then be joined (e.g., by pinching or crimping the ends together and/or clipping the ends together). In some implementations, securing the ends of the chordal ring implant 1700 together can be done substantially simultaneously with cinching the chordal ring implant 1700 to cinch the chords together.

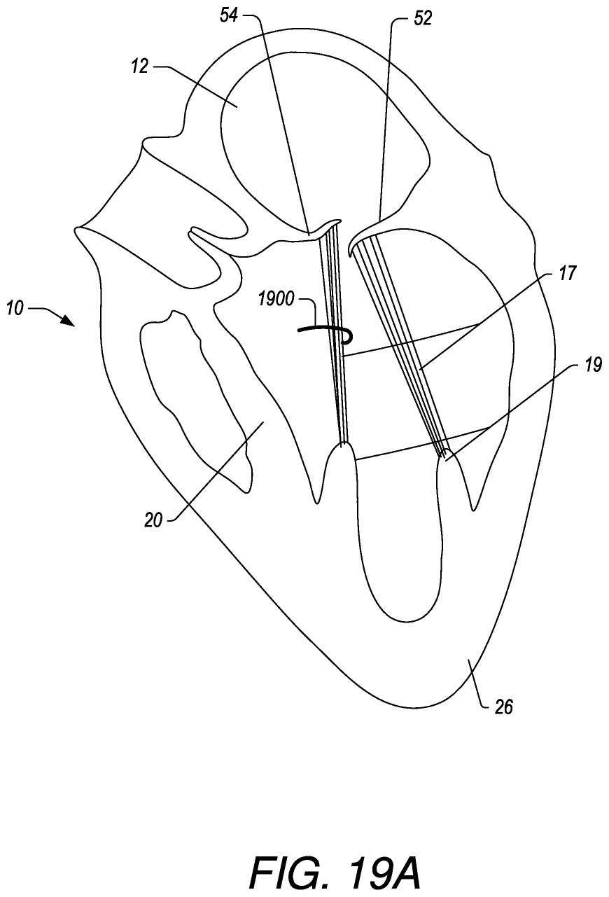

FIGS. 18A and 18B illustrate a chordal clip 1800 configured to cinch elongated chords from the side, thereby reducing leaflet prolapse and/or other issues. The chordal clip 1800 can be configured to pull elongated chords, as shown in FIG. 18A. The chordal clip 1800 can be configured to secure the excess portion to the side to effectively shorten the elongated chords, as shown in FIG. 18B. The chordal clip 1800 can be configured to pinch one or more elongated chords to reduce their effective length. In the pinched state, the chordal clip 1800 can be transitioned to a deployed configuration wherein the chordal clip 1800 tightens and secures the pinched portion of the chords. In some implementations, the chordal clip 1800 comprises a clamp, clip, suture, hook, staple, or the like that is configured to secure the one or more elongated chords by grasping, lassoing, clamping, clipping, or the like.

The chordal clip 1800 can be delivered to the left ventricle 14 via a transcatheter procedure. For example, a transapical approach can be used to deliver the chordal clip 1800 to the left ventricle 14. In the left ventricle 14, the delivery device can secure a portion of the chords to pull to the side. Once pulled to the side, the chordal clip 1800 can be secured to the pulled or pinched portion of the chords to reduce their length, thereby reducing leaflet prolapse and/or other issues.

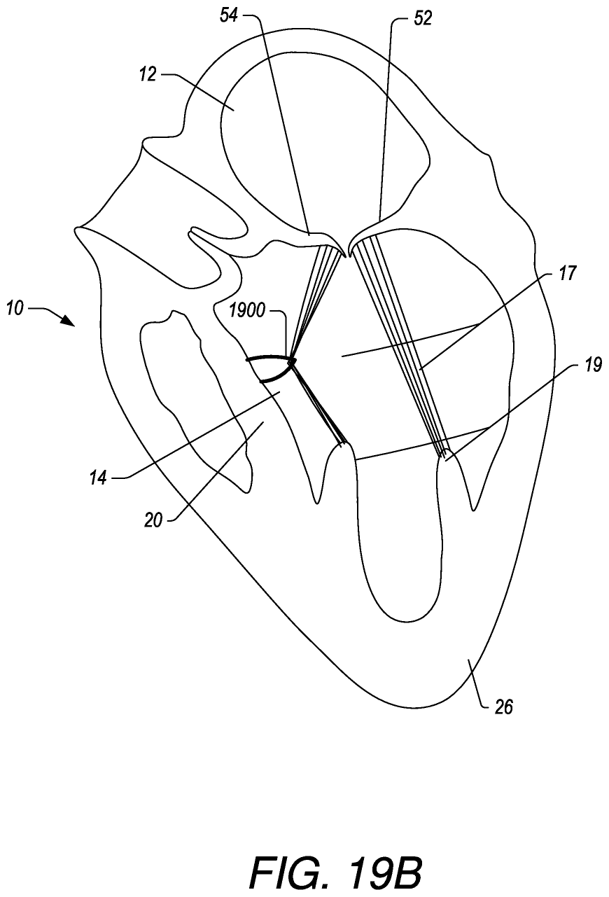

FIGS. 19A and 19B illustrate a staple implant 1900 configured to gather excess portions of elongated chords and to secure the elongated chords to the ventricle wall to effectively shorten the elongated chords. This can reduce leaflet prolapse and/or other issues caused by elongated chords. The staple implant 1900 can be configured to pull elongated chords, as shown in FIG. 19A. The staple implant 1900 can be configured to secure the excess portion to the ventricle wall to effectively shorten the elongated chords, as shown in FIG. 19B. The staple implant 1900 can include anchors, barbs, hooks, or the like to secure end portions of the staple implant 1900 to the ventricle wall. In some implementations, the staple implant 1900 includes a suture that extends between a first anchor and a second anchor.

The staple implant 1900 can be delivered to the left ventricle 14 via a transcatheter procedure. For example, a transapical approach can be used to deliver the staple implant 1900 to the left ventricle 14. In the left ventricle 14, the delivery device can secure a portion of the chords to pull to the side. Once pulled to the side, the staple implant 1900 can be wrapped around the pulled chords and the ends of the staple implant 1900 can be secured to the ventricle wall to reduce the effective length of the chords, thereby reducing leaflet prolapse and/or other issues.

### Sterilization

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Additional Embodiments

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed. Further, the treatment techniques, methods, operations, steps, etc. described or suggested herein can be performed on a living animal (e.g., human, other mammal, etc.) or on a non-living simulation, such as on a cadaver, cadaver heart, simulator (e.g., with the body parts, tissue, etc. being simulated), anthropomorphic phantom, etc.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems and are not limited to the methods and systems described above, and elements and acts of the various implementations described above can be combined to provide further implementations. Accordingly, the novel methods and systems described herein may be embodied in a variety of other forms; furthermore, various omissions, substitutions, and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. A device for treating a native valve, the device comprising:
a first free-edge clipping implant configured to attach to a free edge of a first leaflet;
a second free-edge clipping implant configured to attach to a free edge of a second leaflet;
a cinching mechanism configured to pull the first free-edge clipping implant and second free-edge clipping implant toward the cinching mechanism; and
one or more sutures joining the first free-edge clipping implant and the second free-edge clipping implant to the cinching mechanism,
wherein activation of the cinching mechanism tensions the one or more sutures to reduce a distance between the first and second free-edge clipping implants to approximate the second leaflet and the first leaflet to reduce valvular regurgitation.

2. The device of claim 1, wherein the cinching mechanism comprises a spooling component configured to adjust tension on the one or more sutures to lengthen and shorten the one or more sutures relative to the cinching mechanism.

3. The device of claim 2, wherein the spooling component includes a wheel that is configured to rotate to spool the one or more sutures within or around the spooling component.

4. The device of claim 2, wherein the cinching mechanism comprises a locking component configured to lock the first free-edge clipping implant and the second free-edge clipping implant in place or to lock the one or more sutures in place.

5. The device of claim 4, wherein the locking component interacts with the spooling component to control spooling to adjust tension on the one or more sutures.

6. The device of claim 1, wherein the first free-edge clipping implant and the second free-edge clipping implant each include a clip configured to latch onto the free edge of a respective leaflet.

7. The device of claim 1, wherein, during delivery, the cinching mechanism is configured to shorten the one or more sutures so that the first free-edge clipping implant and the second free-edge clipping implant are adjacent to the cinching mechanism.

8. The device of claim 7, wherein, after delivery to the native valve and prior to attaching the first free-edge clipping implant and the second free-edge clipping implant to respective leaflets, the cinching mechanism is configured to lengthen the one or more sutures.

9. The device of claim 8, wherein, after attaching the first free-edge clipping implant and the second free-edge clipping implant to respective leaflets, the cinching mechanism is configured to tension the one or more sutures to approximate the second leaflet and the first leaflet.

10. The device of claim 9, wherein the cinching mechanism is configured to be operated by a delivery device that delivered the device to the native valve to tension the one or more sutures.

11. The device of claim 1 further comprising a third free-edge clipping implant configured to attach to a free edge of the first leaflet or the second leaflet, the one or more sutures joining the third free-edge clipping implant to the cinching mechanism.

12. The device of claim 1, wherein the device is delivered via a transcatheter procedure to the native valve.

13. A device for treating a native valve, the device comprising:
a spacing material configured to be implanted between a first leaflet and a second leaflet;
a first paddle coupled to the spacing material, the first paddle comprising a first securing mechanism to secure a portion of the first leaflet to the first paddle; and
a second paddle coupled to the spacing material, the second paddle comprising a second securing mechanism to secure a portion of the second leaflet to the second paddle,
wherein each paddle is configured to extend and to retract from the spacing material to attach to an edge of a respective leaflet, each paddle having an independently adjustable length to enable each paddle to secure the respective leaflet to the spacing material to reduce prolapse, flail, or valvular regurgitation.

14. The device of claim 13, wherein the first securing mechanism and the second securing mechanism each comprise hooks.

15. The device of claim 13, wherein a length of each paddle is independently adjusted by manipulating elements at a proximal end of a delivery device.

16. The device of claim 13, wherein the first paddle is configured to secure a middle portion of the first leaflet and the second paddle is configured to secure a middle portion of the second leaflet.

17. The device of claim 13, wherein, in a deployed configuration, an edge of the first leaflet is configured to be secured by the first securing mechanism of the first paddle and an edge of the second leaflet is configured to be secured by the second securing mechanism of the second paddle.

18. A device for treating a native valve, the device comprising:
a clip implant configured to be implanted on an atrial side of a leaflet, the clip implant configured to secure an excess portion of the leaflet to reduce leaflet prolapse and/or valvular regurgitation.

19. A device for treating a native valve, the device comprising:
a first magnetic implant secured to a first leaflet; and
a second magnetic implant secured to a second leaflet, magnetic forces between the first magnetic implant and the second magnetic implant sufficient to reduce prolapse, flail, or valvular regurgitation.

20. A device for treating a native valve, the device comprising:
a tube for drawing in an excess portion of a leaflet;
a cauterizing element configured to excise the excess portion of the leaflet; and
a clip configured to clip the cauterized portion of the leaflet.
